(19) United States
(12) Patent Application Publication
(10) Pub. No.: US 2020/0023073 A1
(43) Pub. Date: Jan. 23, 2020

(54) A NEW DRUG DELIVERY SYSTEM FOR TREATMENT OF DISEASE

(71) Applicant: SINTEF TTO AS, TRONDHEIM (NO)

(72) Inventors: YRR MORCH, TRONDHEIM (NO); RUNE HANSEN, RANHEIM (NO); RUTH SCHMID, TILLER (NO); HEIDI JOHNSEN, TRONDHEIM (NO); PER STENSTAD, TRONDHEIM (NO); ANDREAS ASLUND, TILLER (NO); SOFIE SNIPSTAD, TRONDHEIM (NO); SIGRID BERG, TILLER (NO); EINAR SULHEIM, TRONDHEIM (NO); CATHARINA DAVIES, TRONDHEIM (NO)

(21) Appl. No.: 16/338,152

(22) PCT Filed: Sep. 29, 2017
(86) PCT No.: PCT/EP2017/074798

§ 371 Date: Mar. 29, 2019

(30) Foreign Application Priority Data
Sep. 29, 2016 (NO) 20161568
Jun. 21, 2017 (NO) 20171014

(51) Int. Cl. A61K 47/69; A61K 41/00; A61K 9/51; A61P 35/00; A61K 9/50; A61K 31/337; A61K 9/00; A61M 37/00

(52) U.S. Cl. CPC A61K 47/6933; A61K 41/0028; A61K 9/5192; A61K 47/6925; A61K 9/5146; A61M 37/0092; A61K 9/5089; A61K 31/337; A61K 9/0009; A61K 9/5138; A61P 35/00

(57) ABSTRACT

The present invention is generally directed to improvements in the treatment of cancer and diseases in the central nervous system. A new drug delivery system is provided, method for producing it and medical uses.

### TECHNICAL FIELD OF THE INVENTION

The present invention is generally directed to improvements in the treatment of cancer, cancerous tumors and diseases in the central nervous system. A new drug delivery system is provided, method for producing it and medical uses.

### BACKGROUND OF THE INVENTION

Cancer is a group of diseases involving abnormal cell growth with the potential to invade or spread to other parts of the body. This malignant behavior often causes invasion and metastasis to second locations. Cancer is a major cause of mortality in most industrialized countries. The standard treatments include surgery, chemotherapy, radiation, laser and photodynamic therapy, alone or in combination. In addition, immunotherapy and hormonotherapy have been approved for certain types of cancer. Surgical intervention is used to remove macroscopic tumors and irradiation of the tumor site to treat the remaining microscopic tumors. Chemotherapy is used to attack any residual or non-resectable disease, at either the surgical site or elsewhere in the body. The success rates of the different treatments are depending on the type and stage of the cancer. Although improved in recent years, the prognosis for many types of cancer patients is still poor.

Chemotherapy can be defined as the treatment of cancer with one or more cytotoxic anti-neoplastic drugs (chemotherapeutic agents) as part of a standardized regimen. The term encompasses a variety of drugs, which are divided into broad categories such as alkylating agents and antimetabolites. Traditional chemotherapeutic agents act by killing cells that divide rapidly, a critical property of most cancer cells. This is achieved by impairing mitosis (cell division) or DNA synthesis.

All though chemotherapy is curative for some cancers (such as for example leukemia), it is still ineffective in some and needless in others.

Chemotherapeutic agents are most often delivered parenterally, depending on the drug and the type of cancer to be treated. With traditional parenteral chemotherapy typically only 0.001-0.01% of the injected dose reaches the tumor. Many current chemotherapy drugs unfortunately also have excessive toxicity to healthy tissues and a limited ability to prevent metastases.

Enormous efforts have been put in finding novel tumor-targeting treatments in recent years. Tumors vasculature is generally more ‘leaky’ but suffers from higher interstitial fluid and oncotic pressure that can impede passage of drug throughout the tumor bulk. Uptake of established chemotherapeutics can be highly variable depending on tumor type and such uptake differences may contribute to the variable nature of the therapeutic effect.

Nanoparticles (NPs) as carriers for anti-cancer drugs offer great potential for such targeted cancer therapy as a certain accumulation in the tumor is observed due to the enhanced permeability and retention effect (EPR effect). Still, the uptake of NPs in tumors is relatively low and the distribution heterogeneous. Thus, the nanomedicine field has so far shown limited impact. The indicated EPR effect, on which the nanomedicine field largely relies, has mainly been studied in animal tumor models and there is limited experimental data from patients. The EPR effect shows significant heterogeneity within and between tumor types and there is currently an ongoing debate within the oncological and nanomedicine communities regarding the EPR effect in humans. Novel treatment concepts, enhancing or bypassing the EPR effect are of high clinical interest.

It is known that gas-filled microbubbles (MBs), currently in clinical use as contrast agents for ultrasound (US) imaging, used in combination with therapeutic low-frequency US can locally increase the vascular permeability. This is achieved by inducing an “artificial EPR effect” by loosening up or making pores through tight junctions for paracellular uptake, increased endocytosis and/or transcellular transport from sonoporation. However, commercially available MBs optimized for US imaging have very thin shells (2-20 nm), are fragile and have short blood circulation time (around 1 min). Their application in a drug delivery system to enhance uptake of chemotherapeutic agents to cancerous tissues and tumors is thus limited.

Accordingly, there is a need for an improved drug delivery system, which can increase the vascular permeability and enhance uptake of therapeutic agents in tumors.

Recent work has also been motivated to address the issues of drug delivery across the blood-brain barrier (BBB) to target sites in the central nervous system. Tight vascular endothelial junctions that inhibits the passage of larger molecules to the tissue space characterize the blood brain barrier. Brain delivery of drugs is hindered by the BBB, an interface at brain endothelium that protects the brain and maintains its homeostasis, but also restricts the passage of 98% of small and virtually all large molecular drugs.

Nanoparticles (NPs) can offer numerous benefits in drug delivery due to their high drug loading capacity, incorporation of poorly soluble drugs and novel therapeutics such as peptides and oligonucleotides, functionalization for sustained and controlled release and combination of therapeutics with imaging. In the case of solid tumors, nanoparticles can also benefit from the enhanced permeability and retention effect, whereby NPs are retained in the tumor due to its leaky neovasculature and reduced lymphatic drainage. The BBB, however, is a formidable obstacle for NPs as well, and their brain delivery can benefit from versatile BBB opening techniques. Thus, there is a need to explore the potential use of nanoparticles in drug delivery to the brain.

The most basic form of ultrasound/microbubble mediated drug delivery is administration of a microbubble formulation together with a systemically administered drug. An example of such an approach has recently entered clinical trials [Kotopoulis et al, Med Phys., 40(7) (2013)], where the commercial US contrast agent Sono Vue (Bracco Spa.) is co-administered with Gemcitabine followed by US irradiation for treatment of pancreatic cancer.

In addition to the co-administration approach, several other microbubble technologies are explored for drug delivery [Geers et al, Journal of Controlled Release 164 (2012) 248-255]. Examples are drug-loaded microbubbles, in situ formed microbubbles from nanodroplets and targeted microbubbles. The first clinical phase I trial combining focused ultrasound (FUS) and MBs with chemotherapy has already been reported, where 10 patients with inoperable, locally advanced pancreatic cancer received an infusion of gemcitabine, followed by SonoVue injected intravenously during US treatment (Georg Dimcevski, et al. 2016). Over the years, however, it has been recognized that all these approaches have fundamental limitations, which have effectively hindered a transition to clinical practice. Perhaps the most limiting is the amount of drug that can be incorporated into microbubble systems. In addition, for attachment and/or incorporation of the drug load into the microbubble systems, chemical modification of the drug may be required, with potential changes to biological activity.

Accordingly, there is a need for novel multifunctional drug delivery systems.

The invention is the first successful demonstration of a novel multifunctional drug delivery system comprising gas-filled microbubbles associated with nanoparticles in therapy. As demonstrated herein the system is for use in therapy, such as in treatment of cancer and diseases in the central nervous system. The delivery-system is used in combination with ultrasound to facilitate the delivery of nanoparticles. Enhanced uptake of nanoparticles at the target site (such as in tumors or target sites in the brain) is achieved by applying an acoustic field, such as generated by focused ultrasound.

### Definitions

The term ‘microbubble, (MB)’ is used herein to describe microbubbles with a diameter in the range from 0.5 to 30 microns, typically with a mean diameter between 1 to 6 μm.

The term ‘nanoparticle, (NP)’ is used herein to describe particles or capsules with linear dimensions less than 800 nm.

The terms “microbubble associated with nanoparticles” and “nanoparticles associated with microbubbles” are used herein to describe in what way the nanoparticles interact with the microbubble interface. The term “associated with” as used in connection with this include association by any type of chemical bonding, such as covalent bonding, non-covalent bonding, hydrogen bonding, ionic bonding or any other surface-surface interactions.

The terms “systemic administration” and “administrated systemically” are art-recognized terms and include routes of administration of a substance into the circulatory system so that the entire body is affected.

The terms “parenteral administration” and “administered parenterally” are art-recognized terms, and include modes of administration other than enteral and topical administration, such as injections, and include without limitation intravenous, intramuscular, intrapleural, intravascular, intrapericardial, intraarterial, intrathecal, intracapsular, intraorbital, intracardiac, intradennal, intraperitoneal, transtracheal, subcutaneous, subcuticular, intraarticular, subcapsular, subarachnoid, intraspinal and intrastemal injection and infusion.

The term “target site” and “disease site” are used interchangeably herein to describe the tissue to be treated. It can independently be cancerous tissue, tumors, such as solid tumors, gliomas, such as aggressive glioblastomas, or other diseases in the central nervous system.

The term “release site” is used herein to describe the site wherein an acoustic field is generated to facilitate the release of the nanoparticles and hence the delivery of nanoparticles and therapeutic agent to the target site.

The term “free nanoparticles” describes nanoparticles that are non-associated with the microbubbles.

The term ‘surfactant’ is used in herein for chemical compounds that lower the surface tension between two liquids, or between a gas and a liquid, e.g. used as a stabilizer in a dispersion of microbubbles. ‘Acoustic field’ is the term used to describe the area where the focused ultra-waves are applied, hence the area of exposure or US-treatment. The acoustic field generates “thermal and non-thermal mechanisms”. “Non-thermal mechanisms” include cavitation, vibrations and oscillations.

“High intensity focused ultrasound, (HIFU)” or “focused ultrasound, (FUS)” refers to the medical technology that uses an acoustic lens to concentrate multiple intersecting beams of ultrasound on a target. Each individual beam passes through tissue with little effect but at the focal point where the beams converge, the energy can have useful thermal or mechanical effects. HIFU or FUS is typically performed with real-time imaging via ultrasound or MRI to enable treatment targeting and monitoring (including thermal tracking with MRI).

The term ‘cavitation’ is used to describe the process where MB expand and compress upon exposure to US in the acoustic field. Ultrasound waves propagate through high- and low-pressure cycles, and the pressure differences make the MBs expand during the low-pressure phase and compress during the high-pressure phase. This oscillation can be stable for several cycles (stable cavitation), but it can also end in more or less violent collapse of the MBs (inertial cavitation), depending on the pressure amplitude and frequency. Cavitation-related mechanisms include microstreaming, shock waves, free radicals, microjets and strain. The acoustic radiation force produced by the ultrasound wave can also push MBs towards the vessel walls.

The term “sonoporation”, or “cellular sonication”, is used herein to describe the use of sound (typically ultrasonic frequencies) for modifying the permeability of the cell plasma membrane. Sonoporation employs the acoustic cavitation of microbubbles, thus enhancing the delivery of nanoparticles to tumors and/or at the release site. As used herein, the term “drug delivery” is understood to include the delivery of drug molecules, therapeutic agents, diagnostic agents, genes, and radioisotopes.

The term “pharmaceutical composition” used in this text has its conventional meaning, and are in particular in a form suitable for mammalian administration, especially via parenteral administration, such as injection.

The term “therapeutic agent” is meant to include every active force or substance capable of producing a therapeutic effect. The terms “chemotherapeutic agent” and “anti-cancer drugs” are used interchangeably throughout the description.

The term “diagnostic agent” is used to described substances used to reveal, pinpoint, and define the localization of a pathological process.

The term “pharmaceutically acceptable” as used herein denotes that the system or composition is suitable for administration to a subject, including a human patient, to achieve the treatments described herein, without unduly deleterious side effects in light of the severity of the disease and necessity of the treatment.

The terms “therapy”, “treat,” “treating,” and “treatment” are used synonymously to refer to any action providing a benefit to a patient at risk for or afflicted with a disease, including improvement in the condition through lessening, inhibition, suppression or elimination of at least one symptom, delay in progression of the disease, prevention, delay in or inhibition of the likelihood of the onset of the disease, etc.

The expression “enhanced permeability and retention (EPR) effect” and ‘artificial EPR effect’ are used herein to describe the property by which molecules of certain sizes (typically liposomes, nanoparticles, and macromolecular drugs) tend to accumulate in tumor tissue much more than they do in normal tissue.

The term “blood-brain-barrier” as used herein refers to the highly selective permeability barrier that separates the circulating blood from the brain extracellular fluid in the central nervous system (CNS). The blood—brain barrier is formed by brain endothelial cells, which are connected by tight junctions with an extremely high electrical resistivity.

### SUMMARY OF INVENTION

The present invention is generally directed to improvement in treatment of cancer and cancerous tumors, cancerous tissues and diseases in the brain and/or central nervous system. It has been demonstrated that the delivery system as described may enhance delivery of therapeutic agents to solid tumors, as well as selectively and transiently open the blood-brain barrier.

The present invention includes a nanoparticle filled (or loaded) with a therapeutic agent, a gas-filled microbubble, and the combination of the two. A drug delivery system is disclosed which facilitates the delivery of the therapeutic agent to disease tissue. The system uses ultrasound to induce an acoustic field that covers the diseased area. In the acoustic field, cavitation and/or oscillation can occur. The cavitation or oscillation may cause a possible collapse of the microbubbles. The collapse of gas microbubbles releases the nanoparticles. In the acoustic field, radiation forces produced by the ultrasound waves will act on the microbubbles, and may push them towards the vessel wall before they collapse. Cavitation and collapse can further generate shear stress and jet streams on endothelial cells, which will both, together and independently, improve transport of nanoparticles across the capillary wall.

In a first aspect of the invention, it is disclosed a drug delivery system for use in therapy comprising at least one gas-filled microbubble, a plurality of nanoparticles associated with the at least one microbubble and at least one therapeutic agent associated with at least one of the nanoparticles, wherein the drug delivery system is administered systemically, such as parenterally, and an acoustic field is generated at a release site to mediate the delivery of said nanoparticles and/or the at least one therapeutic agent to a target site.

In different embodiments, the acoustic field may be generated by ultrasound (US), such as focused ultrasound (FUS), or other means known to the skilled person. The acoustic field causes cavitation, oscillation and/or collapse of the gas-filled microbubbles, thereby facilitating release of the nanoparticles. The cavitation may further improve the transport of nanoparticles across the capillary wall. As such, this novel use enhances the EPR effect.

In another embodiment, the delivery is mediated by radiation force and/or heating, which can also lead to increased transport of nanoparticles and drugs in extracellular matrix in tumor tissue.

In a further embodiment, the delivery is mediated by a combination of ultrasound-induced activation of microbubbles and radiation force and/or heating.

In another embodiment, the microbubble is destroyable upon application of focused ultrasound thereto.

In one embodiment of the invention according to the first aspect, the release site is the same as the target site. An example of such embodiment is when the drug delivery system is for use in treatment of cancer. In this embodiment, an acoustic field is generated at a release site, which can be a solid tumor or tumorous tissue, to mediate the delivery of nanoparticles and/or therapeutic agent to a target site, which can be said solid tumor or tumorous tissue.

In another embodiment, the release site is not the same as the target site. An example of this is when the drug delivery system is for use in treatment of diseases in the central nervous system. In this embodiment, the acoustic field is generated at a release site, which can be a blood brain barrier, to mediate the delivery of nanoparticles and/or therapeutic agent to a target site, which can be a disease site in the central nervous system, such as a brain tumor (e.g. glioblastomas) or other disease sites in the brain. In cases, wherein the target site is a solid tumor, the release site may be a part of the target site. In such embodiments, only a part of the target site (e.g. the tumor) is exposed to ultrasound, which generate the acoustic field, upon which the release and enhanced uptake of drug-loaded NPs to the target site is facilitated. Accordingly, the drug delivery system according to the invention is multimodal and multifunctional, and constitutes a novel medical use for treatment of cancer, in particular solid tumors, and brain tumors, as well as other diseases in the central nervous system.

In certain embodiments, the nanoparticles may be surface-associated to the microbubble and covering at least a part of the microbubble surface, optionally the at least one of the nanoparticles are polymeric, such as poly(alkylcyanoacrylate) (PACA) nanoparticle. In preferred embodiments, the PACA-particle is a poly(isohexylcyanoacrylate) or a poly(ethyl butyl cyanoacrylate).

According to one embodiment of the first aspect of the invention, the therapeutic agent is loaded within the nanoparticles. Optionally, the nanoparticles may also contain co-stabilizers.

In another embodiment, the drug delivery system according to the first aspect further comprises free nanoparticles and one or more therapeutic agent associated with the free nanoparticle. In certain embodiments, the nanoparticles associated with the microbubble are the same kind of nanoparticles as the free nanoparticles, and both may be filled with at least one therapeutic agent.

According to another embodiment, the surface-associated polymeric nanoparticles stabilizes the microbubble. The stabilizing of microbubbles by the nanoparticles will influence the possible circulation time of the microbubbles in blood.

In certain embodiments, the drug delivery system according to the invention may further optionally comprise at least one or more targeting agents, a pharmaceutically acceptable carrier, and the nanoparticles may further be coated with a hydrophilic polymer such as polyethylene glycol (PEG).

In certain embodiments, the mean diameter of microbubble with surface-associated polymeric nanoparticles is in the range 0.5 to 30 μm.

The therapeutic agent is in certain embodiments a chemotherapeutic agent or a chemopotentiator.

According to further embodiments, the microbubble may be filled with a gas selected from the group consisting of: perfluorocarbon, air, N2, O2, CO2.

In an alternative aspect, the drug delivery system according to the first aspect is a composition.

In a second aspect, the invention also includes a method for preparing a drug delivery system for use in therapy according to the first aspect of the invention, comprising the steps of:
a) Synthesizing the nanoparticles to be loaded with the therapeutic agent and/or contrast agent.
b) Adding nanoparticles to a solution comprising a surface-active substance.
c) Mixing the solution with gas to obtain gas-filled bubbles.

According to one embodiment of the method, the microbubbles are stabilized by self-assembly of nanoparticles in the gas-water interface.

In certain embodiments of the method, the solution in c) is mixed with gas for a desired time, such as from about 2 seconds to 60 minutes, preferentially 1 to 10 minutes, and/or desired speed, such as about 500 to 50 000 rpm when ultraturrax mixing is used, preferentially 1000 to 30000 rpm to obtain microbubbles of desired size.

According to certain embodiments of the method, the surface-active substance in the solution in step b) is selected from the group consisting of a protein or a lipid or a polymer or a surfactant.

A third aspect of the invention is a composition comprising a gas-filled microbubble, a plurality of nanoparticles associated with the microbubble and one or more therapeutic agent associated with one or more of the nanoparticle, wherein the composition further comprises free nanoparticles, i.e. nanoparticles that are non-associated with the microbubbles.

In certain embodiments of this aspect, the plurality of nanoparticles may be surface-associated to the gas-filled microbubble. Further said plurality of nanoparticles may be covering at least a part of the microbubble surface. Optionally the plurality of nanoparticles and/or the free nanoparticles are polymeric, such as poly(alkylcyanoacrylate) (PACA) nanoparticles. In one preferred embodiment the PACA-particles are poly(ethyl butyl cyanoacrylate) nanoparticles.

According to one embodiment of the third aspect of the invention, the therapeutic agent is loaded within the nanoparticles. Optionally, the nanoparticles may also contain co-stabilizers.

In further embodiments, the composition according to the third aspect of the invention is for use in therapy. According to these embodiments, the composition is administered systemically, such as parenterally, and an acoustic field is generated at a release site to mediate the delivery of said nanoparticles and/or the at least one therapeutic agent to a target site. Different features as described according to the first aspect of the invention also applies to the composition according to the third aspect for use in therapy

A last aspect of the invention includes a method of treating cancer comprising administering a drug delivery system according to the first aspect of the invention to a patient in need thereof. In one embodiment, it is disclosed a method of treating diseases in the central nervous system comprising administering a drug delivery system according to the first aspect of the invention to a patient in need thereof.

The therapeutic agent may be loaded within at least one nanoparticle, optionally, the system according to the first aspect of the invention may also comprise nanoparticles loaded with diagnostic agents.

Certain embodiments of the present invention include a method for the treatment of cancer or diseases in the central nervous system, comprising delivering a microbubble with associated nanoparticles to a treatment site of a patient, wherein the at least one nanoparticle is filled with a therapeutic agent. In some embodiments, the method includes applying ultrasound energy to the treatment site. In some embodiments, the disease is cancer, such as breast cancer or cancer in the brain.

Further aspects of the invention is found in the following numbered embodiments:
1. A drug delivery system comprising a gas-filled microbubble, a plurality of nanoparticles associated with the gas-filled microbubble and at least one therapeutic agent associated with at least one nanoparticle for ultrasound-mediated delivery of the nanoparticles and/or the at least one therapeutic agent to a tumorous tissue.
2. The drug delivery system according to numbered embodiment 1, wherein the nanoparticles are surface-associated to the gas-filled microbubble.
3. The drug delivery system according to any one of the numbered embodiments 1-2, wherein the at least one therapeutic agent is loaded within the nanoparticles.
4. The drug delivery system according to any one of the numbered embodiments 1-3, further comprising at least one free nanoparticle and at least one therapeutic agent associated with the at least one free nanoparticle.
5. The drug delivery system according to any one of the numbered embodiments 1-4, wherein the nanoparticles are polymeric.
6. The drug delivery system according to any one of the numbered embodiments 1-5, wherein at least one of the nanoparticles is a poly(alkylcyanoacrylate) (PACA) nanoparticle.
7. The drug delivery system according to any one of the numbered embodiments 1-6, wherein the nanoparticles associated with the gas-filled microbubbles stabilizes the microbubbles.
8. The drug delivery system according to numbered embodiments 1-7, wherein the nanoparticles further comprising at least one targeting agent.
9. The drug delivery system for use according to anyone of the numbered embodiments 1-8, further comprising a pharmaceutically acceptable carrier.
10. The drug delivery system according to any one of the numbered embodiments 1-9, wherein the nanoparticles further are coated with polyethylene glycol (PEG).
11. The drug delivery system according to any one of the numbered embodiments 1-10, wherein the mean diameter of the gas-filled microbubbles associated with nanoparticles is in the range 0.5 to 30 μm.
12. The drug delivery system according to any one of the numbered embodiments 1-11, wherein the therapeutic agent is chemotherapeutic agent or a chemopotentiator.
13. The drug delivery system according to any one of the numbered embodiments 1-12, wherein the gas-filled microbubbles is filled with a gas selected from the group consisting of: air, perfluorocarbon, N2, O2, CO2.
14. The drug delivery system according to any one of the numbered embodiments 1-13, wherein the ultrasound-mediated delivery is mediated by ultrasound, such as focused ultrasound.
15. The drug delivery system according to any one of the numbered embodiments 1-14, wherein the microbubbles is destroyable upon application of focused ultrasound thereto.
16. A method for preparing a drug delivery system according to the numbered embodiments 1-15, comprising the steps of:
   a. Synthesizing the nanoparticles to be loaded with the therapeutic agent.
   b. Adding nanoparticles to a solution comprising a surface-active substance.
   c. Mixing the solution with gas to obtain gas-filled bubbles.
17. A method according to numbered embodiment 16, wherein the microbubbles is stabilized by self-assembly of nanoparticles in the gas-water interface.
18. A method according to any one of the numbered embodiments 16-17, wherein the solution with gas is mixed for a desired time and/or desired speed to obtain microbubbles of desired size.
19. A method according to any one of the numbered embodiments 16-18, wherein the solution in c) is mixed from 2 seconds to 60 minutes, preferentially 1 to 10 minutes.
20. A method according to anyone of the numbered embodiments 16-19, wherein the solution in c) is mixed at 500 to 50 000 rpm, preferentially 1 000 to 30 000 rpm
21. A method according to anyone of the numbered embodiments 16-19, wherein the surface-active substance is a serum, a protein or a lipid or a surfactant.
22. A gas-filled microbubble associated with nanoparticles for use in treatment of cancer, wherein at least one of the nanoparticles is loaded with a therapeutic agent and delivery of the nanoparticles and/or therapeutic agent to tumorous tissue is facilitated by an acoustic field, such as by ultrasound.
23. Use according to numbered embodiment 22, wherein the nanoparticles is surface-associated to the microbubble and covering at least a part of the microbubble surface.
24. Use according to any one of the numbered embodiments 22-23, wherein the surface-associated nanoparticles stabilizes the microbubble.
25. Use according to anyone of the numbered embodiments 22-24, wherein the acoustic field causes cavitation, oscillation and/or collapse of the gas-filled microbubbles.
26. Use according to anyone of the numbered embodiments 22-25, wherein the cavitation improves the transport of nanoparticles across the capillary wall.
27. Use according to numbered embodiments 22-26, wherein the surface-associated nanoparticles further comprising at least one or more targeting agents.
28. A composition for use in treatment of cancer comprising a gas-filled microbubble, a plurality of nanoparticles associated with the microbubble and one or more chemotherapeutic agent associated with one or more of the nanoparticle.
29. A composition for use according to numbered embodiment 28, wherein the composition further comprises at least one free nanoparticles and one or more chemotherapeutic agent associated with said nanoparticle.
30. A composition for use according to numbered embodiment 28, wherein the composition comprises a drug delivery system according to any one of the numbered embodiments 1-15.
31. A method of treating cancer comprising administering a drug delivery system according to the numbered embodiments 1-14 to a patient in need thereof.

### BRIEF DESCRIPTION OF DRAWINGS

FIG. 1: Size distribution of PEGylated cabazitaxel-loaded PIHCA NPs as measured by dynamic light scattering. The drug loading is 10.7 wt %.

FIG. 2: Histogram showing size distribution of MBs stabilized by PEGylated cabazitaxel-loaded PIHCA NPs as measured by light microcsopy and image analysis.

FIG. 3: Electron microscopy image of microbubble with surface-associated nanoparticles

FIG. 4. The size and zetapotential of the NPs were approximately 170 nm and −1 mV, respectively. Cellular uptake in the breast cancer cell line was confirmed by CLSM (A). The NPs were imaged by encapsulating a fluorescent dye (red). From quantification by FCM, 90% of the cells had taken up NPs by endocytosis after 3 h incubation (B).

FIG. 5. In vivo circulation half-life of the PEGylated NPs was found to be 136 minutes (n=5 animals) (A). An exponential decay on the form of 206160.9e^{-0.0051x} fitted the data with R²=0.67 and p-values ≤0.0001. The MBs stabilized by the self-assembled NPs had a size of approximately 3 μm, and were found to be suitable for in vivo contrast enhanced US imaging and image guided drug delivery. Contrast enhancement due to inflow and circulation of bubbles in a tumor imaged by ultrasound(B).

FIG. 6: The biodistribution of NPs 6 h post injection. An example of organs and tumor from one animal is shown (A). Quantification of accumulation in organs and tumors is shown as mean and standard deviation (n=10 animals, n=5 for brain) (B). Autofluorescence from non-treated organs and tumor is shown from one animal.

FIG. 7: 87% of the dose can be found in these organs, tumor and brain. The rest is likely found in urine, stool, skin, muscle and other tissues. The majority of the dose is located in the liver and spleen, and about 1% of the dose is located in the tumor (Corresponds well with the reported 0.7% median)

FIG. 8: An example of a CLSM tile scan from an entire tumor section, showing NPs in red (A). The number of pixels with fluorescence from NPs was quantified in tile scans from each animal (B). Similar results were seen when pixel intensities were measured. No effect of stable cavitation was found, whereas the violent collapse of MBs increased the delivery of NPs to tumors, and the uptake increased with increasing MI.

FIG. 9: Analysis of sections, uptake of PIHCA NPs.

FIG. 10: Except for the highest MI (G7), which caused substantial visual hemorrhage, evaluation of HES stained tumor sections showed that all FUS treatments were considered safe. Example of an overview image (A), and representative images of non-treated and treated tissue are shown (B and C, respectively).

FIG. 11: The microdistribution of NPs in the tumors 2 h post treatment was imaged using CLSM. Representative examples from the control group that did not receive any ultrasound treatment (A) and a group that was treated with high pressure (B). Blood vessels are shown in green and nanoparticles in red. An increased delivery of NPs is observed in the treated group (G6) compared to the control group. Distribution of fluorescent dye in tumors with (b) and without (a) applying ultrasound. 250 times more drugs in b) than in a).

FIG. 12: Probing the intracellular degradation of poly (alkyl cyanoacrylate) nanoparticles using confocal microscopy. Measuring the drug release intracellularly

FIG. 13: Uptake of nanoparticles in cells, in vitro.

FIG. 14: Viability of MDA-MB-231 cells (human epithelial, mammary FIG. 15: Uptake of MRI contrast agent in brain. This specific agent will normally not pass the BBB. Thus, the results illustrate transient BBB opening.

FIG. 16: FUS-mediated BBB disruption and transport of NPs across the BBB. a) BBB opening mediated by FUS in combination with the PIHCA-MB platform. b) transport of PIHCA NPs across the BBB following FUS exposure. Red—PIHCA NPs, Green—blood vessels.

FIG. 17: Weight of the animals as a function of time is shown as average and standard deviation for the three different treatment groups. n=4 animals pr group. Day 0 is the day of implantation of tumor cells. Treatments were done at day 21 and 29.

FIG. 18: Tumor volume as a function of time is shown as average and standard deviation for the three different groups. Group 1: Control, saline. Group 2: Microbubbles associated with nanoparticles and the cytostatic drug (cabazitaxel). Group 3: Ultrasound and microbubbles associasted with nanoparticles and the cytostatic drug. n=4 animals pr group. Day 0 is the day of implantation of tumor cells. Treatments were done at day 21 and 29

FIG. 19: Tumor volume at day 35 after tumor cell implantation for the three different treatment groups, n=4 animals pr group. Mean and standard deviation is shown

FIG. 20: A Schematic illustration of enhanced drug delivery to tumor tissue by the use of focused ultrasound and nanoparticle stabilized microbubbles.

FIG. 21: Effect study in mice with subcutaneous breast cancer. Tumor volume as a function of time after implantation of cells (day 0). Mice were treated with saline, nanoparticle-stabilized microbubbles (NPMB) with cabazitaxel, or NPMB with cabazitaxel and US. Treatments were performed at day 21 and 29. Data are shown as mean and standard deviation from n=4 animals in each group until day 35, and n=3 animals per group from day 37.

FIG. 22: Effect study in mice with orthotopic breast cancer. Tumor volume as a function of time after implantation of cells (day 0). Mice were treated with saline (control), NPMB containing cabazitaxel combined with FUS, or commercial MBs (SonoVue) co-injected with NPs containing cabazitaxel combined with FUS. Mean tumor volume for each of the groups.

### DETAILED DESCRIPTION

The present invention is directed to a multifunctional drug delivery system comprising MBs and a plurality of NPs to be used with FUS-mediated drug delivery. It is an innovative drug delivery system allowing for controlled and enhanced delivery of anticancer agents to tumors with the aid of focused US (FUS). Accordingly, the drug delivery system is for use in therapy

The drug delivery system according to the invention comprises gas-filled microbubbles associated with nanoparticles, wherein at least one of the nanoparticles is loaded with a therapeutic agent and delivery of the nanoparticles to target sites, such as tumors, is facilitated by an acoustic field generated by ultrasound. The delivery system is for systemic administration. Accordingly, the delivery system is administered systemically, while the delivery of nanoparticles to the target site is facilitated locally by the aid of FUS. The gas-filled MBs associated with NPs loaded with at least one therapeutic agent may be used in treatment of cancer. In particular, the MBs associated with NPs, according to the invention, are for use in treatment of solid tumors, including tumors in the brain. The gas-filled MBs associated with NPs loaded with at least one therapeutic agent may also be used in therapy, such as for treatment of tumors as glioma. By associating the NPs with MBs and using the system according to the invention, it is possible to enhanced the uptake and effect of the therapeutic agent.

In one embodiment, the gas-filled MBs is stabilized by NPs. The NPs stabilize the gas/water interfaces by self-assembly at the MB surface, thus resulting in very stable MBs. One advantage of the nanoparticle-stabilized MBs according to one embodiment of the invention is thus increased stability and shelf-life.

Without being bound by theory, the association between the NPs and MBs may be the result of the formation of so-called Pickering emulsions. It is known that solid particles with intermediate hydrophobicity can adsorb strongly at the interface between immiscible fluids such as oil—water, enabling the formation of Pickering emulsions, i.e. emulsions stabilized by solid particles of nano- or micrometer size. In the same manner, solid particles can be used to stabilize gas—water interfaces. However, few materials inherently possess the sufficient balance of hydrophobicity and hydrophilicity essential for particle-stabilizing action. As described herein, the NPs as included in the delivery system according to the invention can be used to stabilize the gas—water interface by self-assembly at the MB surface. According to this embodiment, the MBs are formed by self-assembly of NPs into a shell. The result is very stable MBs. Such nanoparticle-stabilized microbubbles are shown to have long shelf life.

The delivery of nanoparticles and the therapeutic agent to the target site is enhanced by applying ultrasound. The ultrasound waves induce an acoustic field that covers the diseased area. With ultrasound applied locally at the release site (e.g. the tumor or the BBB), small pores in the blood vessel will transiently be formed. The acoustic field generated by ultrasound will cause the bubbles to oscillate and collapse, leading to release of individual NPs. It is known from prior art that FUS for therapeutic purposes can be employed to create thermal or mechanical effects such as cavitation and radiation force in tissue (Pitt W G, Husseini G A, Staples B J: Ultrasonic drug delivery—a general review. Expert Opin Drug Deliv 2004, 1:37-56. And Frenkel V: Ultrasound mediated delivery of drugs and genes to solid tumors. Adv Drug Deliv Rev 2008, 60:1193-1208). Cavitation is the creation and oscillation of gas bubbles upon exposure to the acoustic field. At relatively low pressures, the acoustic pressure waves will cause stable cavitation of the MBs; continuous oscillation with expansion and compression inversely proportional to the ultrasound (US) pressure. This results in microstreaming in the vasculature, and shear stresses on the blood vessel wall when the MBs are in contact with the endothelium, which causes formation of small pores and increases the vascular permeability, and enhances endocytosis. Accordingly, when applying ultrasound, it will cause sonoporation, which enhances the vascular permeability. The drug-loaded NPs that are no longer attached to the MBs may then accumulate in tumor tissue thanks to the enhanced vascular permeability.

The delivery of nanoparticles and the therapeutic agent to tumor tissue and/or cancer cells are enhanced by applying ultrasound or an acoustic radiation force. The ultrasound or acoustic radiation force induce an acoustic field that covers the diseased area. With ultrasound applied locally at the tumor, small pores in the blood vessel will transiently be formed. The acoustic field generated by ultrasound will cause the bubbles to oscillite and collapse, leading to release of individual NPs. The ultrasound also causes sonoporation, which enhances the vascular permeability. Drug-loaded NPs may then accumulate in tumor tissue thanks to the enhanced vascular permeability.

The present invention is a delivery system for use in therapy, and this is the first demonstration of therapeutic effects in an in vivo animal model. Upon administering the drug delivery system systemically, US is applied at the release site to mediate the delivery of said nanoparticles and/or the at least one therapeutic agent to the target site.

Without being bound by theory, the effects observed in the described study may be due to several mechanisms:
1. It is known that tumors have a leaky vasculature and nonfunctional lymphatics. This result in the enhanced permeability and retention (EPR) effect, which allows NPs to selectively extravasate and accumulate in tumors, while the healthy tissue is less exposed. Accordingly, simply by incorporating drugs in NPs one can potentially improve pharmacokinetics, increase efficacy and reduce toxicity of the drug compared to conventional chemotherapy, resulting in reduced dose-limiting side effects
2. When ultrasound is applied at the release site, it causes oscillation of the gas bubbles, thereby enhancing the EPR-effect even further. At relatively low pressures, the acoustic pressure waves will cause stable cavitation of the MBs. Stable cavitation is characterized by sustained bubble radius oscillation about its equilibrium. This generates microstreaming, fluid flow around the MBs. Resulting shear stresses on the blood vessel wall when the MBs are close to or in contact with the endothelium, can cause formation of small pores and increase the vascular permeability, and enhance endocytosis
3. Ultrasound will by itself also push the NPs into the tumor.
4. The enhanced EPR effects will cause any free NPs to accumulate in tumor tissue.

At higher pressures, the oscillation will increase in amplitude, become non-linear and result in a violent collapse of the bubble. This inertial cavitation will lead to the formation of shock waves and jet streams in the vasculature, which can create temporary pores in the capillary wall and in cell membranes (Lentacker I, De Cock I, Deckers R, De Smedt S C, Moonen C T: Understanding ultrasound induced sonoporation: definitions and underlying mechanisms. Adv Drug Deliv Rev 2014, 72:49-64).

The probability of inertial cavitation in a medium is determined by the mechanical index (MI), which is given by the frequency and the peak negative pressure of the US. At intermediate pressures, NP-stabilized MBs will oscillate and collapse, but in a less violent process than in inertial cavitation. Altogether, FUS can thus locally increase the extravasation across the capillary wall and potentially improve penetration through the ECM, thereby improving the uptake and distribution of NPs and drugs at the target site.

In one embodiment of the invention, the delivery system further comprises free nanoparticles, i.e. nanoparticles that are non-associated with the microbubbles, and at least one therapeutic agent associated with the free nanoparticles.

Without being bound by theory, the advantages of this embodiment of the invention is a result of several mechanisms:
MBs in combination with ultrasound create an artificial EPR effect transiently increasing the permeability of blood vessel walls. This enhances the accumulation of freely circulating NPs, i.e. the free NPs that are loaded with at least one therapeutic agent.
NPs associated with MBs (NPMB) will, upon bubble destruction by US, lead to high local deposit of NPs (and hence therapeutic agent), and deeper penetration into tumor tissue

As such, the drug delivery system according to this embodiment of the invention may deposit an even higher concentration of therapeutic agent than MBs associated with NPs alone.

The general principle is that the present invention utilizes nanoparticles (NPs) to deliver drugs. The nanoparticles are typically too large to penetrate healthy blood vessels, but small enough to extravasate the (tumor) blood vessels via the enhanced permeability and retention (EPR) effect or via ultrasound-induced “artificial EPR effect”.NPs according to the invention may be loaded with therapeutic agents, such as anti-cancer agents, and/or diagnostic agents such as contrast agents. In one embodiment, the NPs are biodegradable. Contrast agents can optionally be further incorporated into the NPs for monitoring and follow-up of the NPs. Optionally, the nanoparticles may optionally contain co-stabilizers.

The NPs may typically be of a size from about 1-800 nm, such as about 10-500, preferably about 70-150 nm.

The NPs may further be surface functionalized.

The NPs may further be coated with a hydrophilic polymer such as polyethylene glycol (PEG) to avoid recognition by immune cells. Coating with PEG may further increase blood circulation time.

In another embodiment, the NPs are targeted by targeting moieties. Molecules targeting specific cells may optionally be attached to the NP surface in order to increase the local deposit of NPs at the disease site. The NPs according to the invention is designed for encapsulation of anti-cancer agents. Further, they may successfully be used for producing stabile MBs as described herein. In certain embodiments, the NPs are polymer-based NP, composed of the widely used biocompatible and biodegradable poly(alkyl cyanoacrylate) (PACA) polymer. As demonstrated herein, the NP according to the invention is especially well suited for BBB penetration. In one particular embodiment, the drug-loaded biodegradable NPs is a polymer-based nanoparticle as described in WO 2014/191502.

The NPs may be prepared in a one-step synthesis as described in W02014/191502, with or without targeting moieties. PACAs can encapsulate a range of drugs with high loading capacity, and can easily be further functionalized with polyethylene glycol (PEG). The mean diameter of the MBs associated with a shell of PACA NPs is in the range from 0.5 to 30 μm, such as from 1-10 μm.

In different embodiments poly(butyl cyanoacrylate) (PBCA) NPs, poly(isohexyl cyanoacrylate) (PIHCA) NPs and/or poly(2-ethyl-butyl cyanoacrylate) (PEBCA) may be used. Due to a longer and branched alkyl monomer chain, PEBCA were applied in the study as described in Example 6. PEBCA have a slower degradation rate, which may be therapeutically favorable.

Nanotechnology has started a new era in engineering multifunctional NPs to improve diagnosis and therapy of various diseases, incorporating both contrast agents for imaging and drugs for therapy into so called theranostic NPs. In cancer therapy, encapsulating the drugs into NPs, such as described herein, will improve the pharmacokinetics and reduces the systemic exposure due to the leaky capillaries in tumours. The NPs according to the invention have also been shown to have a potential of treating diseases in the central nervous system (CNS) as they can pass through the BBB. The access of molecules to the CNS is strictly controlled by the specialized and tight junction between the endothelial cells forming the blood vessels constituting the BBB.

In one embodiment, the nanoparticles comprised in the system of the invention is a poly(alkyl cyanoacrylate) (PACA) NP. PACA NPs have shown promise as drug carriers both to solid tumors and across the BBB. This is partly due to the flexibility of the system allowing surface functionalization and drug encapsulation in one step.

Moreover, the degradation and drug release from these nanoparticles (NPs) can be tuned by choosing different monomers. In one embodiment, the NP is prepared by the method as described in WO 2014/191502.

As described herein, the nanoparticles are used in association with MBs. In certain embodiments, the NPs may stabilize the MBs by self-assembly at the MB gas/liquid interface thus forming a stabilizing shell around the MBs. The result is a very stable microbubble with improved technical features. In certain embodiments, the MBs are produced by addition of a further stabilizing agent, such as a surface-active agent. The stabilizing agent may be a surface-active agent chosen from the group of serum, proteins, polymers, lipids or surfactants. The MBs may be produced mixing the solution comprising nanoparticles with a gas by using ultra-turrax, shaking, ultrasound, or other means known to the skilled person. In certain embodiments, the NPs will self-assemble in the gas/liquid interface and form a stabilizing shell around the MBs. In certain embodiments, the nanoparticle-stabilized MBs reduce the fragility of the MBs e compared to commercially available MBs.

In order to improve the uptake and distribution of NPs into diseased tissue, the administration of NPs according to the invention is combined with a treatment facilitating the delivery, such as by applying ultrasound to establish an acoustic field. Without being bound by theory, the hypothesis is that ultrasound is able to improve drug delivery by different mechanisms. In an acoustic field, cavitation, which is the oscillation and possible collapse of gas microbubbles, can occur. Cavitation can then generate shear stresses and jet streams on endothelial cells thereby improving the transport of NPs across the capillary wall. In certain embodiments, the improved extravasation and distribution of NPs in tumours may be achieved by a non-thermal mechanism, however heating and radiation forces may also further enhance the delivery.

In certain embodiments, the present invention comprises three elements:

1. NPs containing the therapeutic agents and contrast agents, alone or in combination.

2. Gas-filled MBs stabilized by the drug-loaded NPs

3. Ultrasound technology for ultrasound-mediated drug delivery using the NP-stabilized MBs

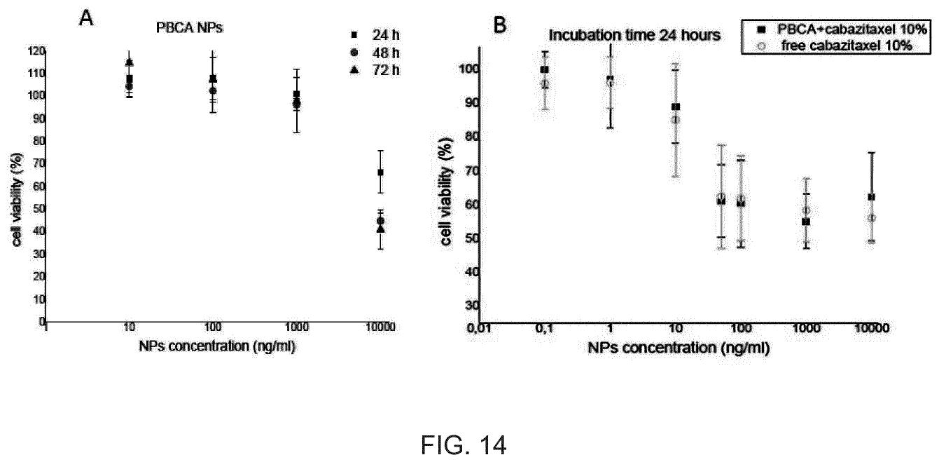

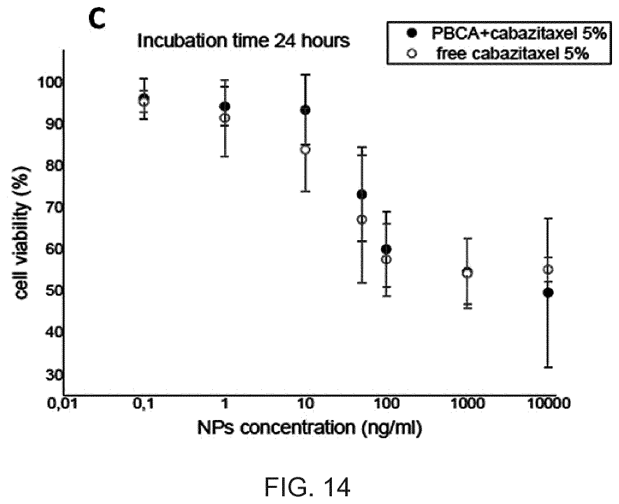

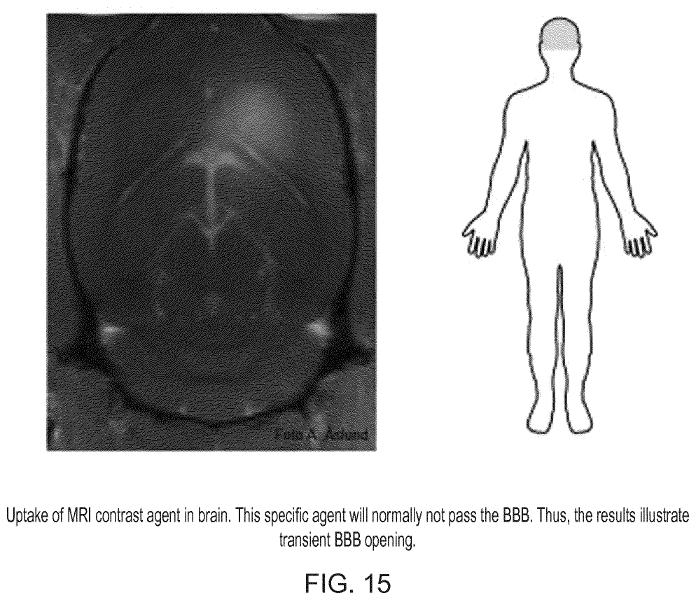

This novel multimodal, multifunctional drug delivery system according to this embodiment of the invention have been shown to improve delivery of therapeutic agents to cancer cells by ultrasound-mediated delivery of NPs. Combining these NP-associated MBs with focused ultrasound results in a higher uptake and improved distribution of the NPs in tumors growing, thus resulting in an improved treatment of cancer. As demonstrated in Example 3 and FIG. 14, the invention results in reduced tumor growth compared to controls.

The new NP-associated MBs can also be used to penetrate the BBB, as documented by magnetic resonance imaging and localization of fluorescently labelled NPs in brain tissue (se FIGS. 15, 16 and 17). Thus, the new NP-associated MB platform demonstrates promising clinical potential in treatment of brain cancer.

Ultrasound and MBs can improve the delivery of non-encapsulated drugs, as recently demonstrated in a clinical study combining ultrasound and co-injection of gemcitabine and commercially available MBs to treat pancreatic cancer. The combination of ultrasound and MBs can also facilitates a transient and local opening of the blood-brain barrier, thereby permitting various drugs to enter the brain and thus treat central nervous system (CNS) disorders. The exact mechanism by which ultrasound and MBs causes blood-brain barrier disruptions is not fully understood, but it is speculated that cavitation i.e.; volume oscillations of MBs in an ultrasound field, might be an important factor.

According to one embodiment of the invention, a mixture of individual drug-loaded NPs and NPs associated with MBs , are injected into the blood stream and will quickly be distributed throughout the entire circulation system. These MBs and free NPs are too large to cross the blood vessel wall of healthy tissue. When entering the acoustic field, applied locally at the tumor site or release site, the MBs will undergo large volume oscillations. During this process, the vascular permeability will be transiently increased due to mechanical stimuli from the oscillating MBs forming small pores in the blood vessel wall. US focused to the release site will also induce bubble collapse, releasing individual NPs from the MB shell for highly targeted treatment. Upon MB destruction, a very high local concentration of drug-loaded NPs is thus obtained. The delivery of the NPs to the target site is thereby facilitated.

The acoustic activity of NP-associated MBs is demonstrated both in vitro and in vivo. As such, they have a great potential in therapeutic applications. It is further shown that US can destroy the MBs, as described herein, thus releasing individual NPs and enhancing model-drug uptake in tumor-bearing mice. The enhanced uptake of model-drug is also demonstrated in cells.

In an experiment where uptake of NPs in cells where studied, the inventors discovered that uptake of PACA NPs in cells were significantly increased when NP-stabilized MBs (also referred to as NPMB) were used compared to co-injection of commercial MBs and PACA NPs or PACA NPs alone. This illustrated that the presence of NPs on the MB surface may further improve efficient delivery of NPs to the disease site and sonoporation, thus contribute to the demonstrated enhanced effects of the system according to the invention.

Further, it is shown that the BBB in rats maybe safely and transiently opened using the novel MBs together with NPs and US. Finally, the effect of MBs associated with NPs is demonstrated in cancer treatment, by the in vivo study described in example 5, 7 and 8. The study demonstrates for the first time the applicability of the described drug delivery system in cancer treatment, as the result demonstrate the ability to significantly reduce tumor growth compared to control. Finally, the applicability of the delivery system for use in treatment of diseases in the central nervous system has been demonstrated in Example 9.

There is a clear need for novel drug-delivery system comprising MBs and NPs with a high drug payload, specifically designed for US-mediated drug delivery applications. Currently, there are no such products on the market. The system according to the invention fills the void and is thus relevant for tumors that are not effectively treated using existing chemotherapeutic technology.

The uniqueness of the invention is its simplicity and versatility, still leading to highly suitable acoustic and biological properties for US-mediated cancer therapy. The advantages of the invention compared to the research systems described today are:
The invention offers a multifunctionality in one simple formulation, which constitute an innovative and advantageous drug delivery system for clinical applications.
The invention can be used separately or simultaneously for US-imaging, diagnosis and therapy
The invention has circulation times significantly longer than commercial MBs.
The invention comprises a combination of individual free NPs and NP-associated MBs, hence allowing for the targeted delivery of very high drug concentrations to tumor tissue
The invention integrates NPs incorporating high payloads of drugs and MBs into one single unit (NP-associated MBs). Integrating NPs and MBs into a single unit is found to have the potential to be much more efficient in US-enhanced tumor uptake as compared to co-injection of NPs and MBs. This is probably caused by a higher concentration of NPs locally in the region of sonication where the MBs are destroyed, in contrast to when NPs and MBs are co-injected intravenously and the NPs are diluted systemically in the blood stream.
The MBs are prepared in a one-step process by self-assembly of NPs at the gas/liquid interphase.
The NPs are also prepared in a one-step process and without the use of organic solvents. This offers a simple, cost-efficient and easy translation to the clinic and into profitable products.

The MBs are associated with thousands of single drug-loaded NPs, as opposed to MBs currently on the market, which are composed of a solid shell of lipids, proteins or polymers. This offers a flexible, yet tough and stable shell, and the ability to release the individual NPs small enough to reach the tumor target and other target sites.

The novel drug delivery system according to the invention clearly addresses the need for novel treatment concepts for enhanced delivery of anti-cancer agents. Further, the invention has the potential to improve treatment of solid tumors significantly, as well as for diseases in the central nervous system. Given the typically poor responses seen with small molecules in solid tumors and the low clinical success up to now with nano-drugs based on the EPR effect, the invention may have a major social impact. Lives may be saved and after-costs of acute and remedial therapy can potentially be greatly reduced. Enhanced drug penetration induced by the invention may affect the necessity of debilitating surgeries. In different embodiments, the invention may particularly be used within a few specific areas of high clinical relevance:
Patients with inoperable cancer
Patients with primary tumors or metastases in the brain. Here there is a strong need for novel delivery techniques, as most anti-cancer drugs will not reach the tumor due to the tight junctions of the BBB.

According to one embodiment, the MBs can be used for contrast enhanced US imaging. The NPs can contain drugs as well as contrast agents, and may be optionally further functionalized with targeting ligands. The NPs may further be coated with a hydrophilic polymer, such as polyethylene glycol (PEG), to improve their circulation time and biodistribution. Accordingly, the invention discloses a highly versatile system.

The chemotherapeutic agent comprised in the nanoparticles may be selected from the group, but are not limited to, the drug classes: Alkylating agents, antimetabolites, cytotoxic antibiotics, topoisomerase inhibitors, anti-microtubule agents or any other known chemotherapeutic agents known to the skilled person.

The cancer treated with the nanoparticles may be solid tumors or cancerous cells. In a particularly preferred embodiment, the cancer is a breast cancer.

The drug delivery system as described herein is for systemic administration. Systemic administration of the drug delivery system as described herein may preferably be achieved by administration into the bloodstream, such as parenteral administration, injection, intravenous or intra-arterial administration.

To achieve successful and sufficient delivery of NPs to the target site, the NPs must circulate in blood for a sufficient amount of time. One particular advantage with the described invention is the improved circulation time of a delivery system wherein the MBs are stabilized by NPs compared to commercially available microbubbles such as Albunex (GE Healthcare), Optison (GE Healthcare), Sonazoid (GE Healthcare), SonoVue (Bracco). The inventors have found that a particular embodiment of the described invention achieve in vivo circulation half life of NPs in an animal model (mice) up to 136 min. This was for instance demonstrated with the use of PEGylated PEBCA.

In vivo circulation of NPs depends on particle material, shape, size, surface chemistry and charge, and it has been demonstrated that circulation time may vary significantly between different NP formulations (Alexis, et al. 2008, Longmire, et al. 2008). To avoid premature degradation and release of payload in blood, NPs that are not delivered to the target should be cleared before the particles release the drug. A common strategy to increase circulation is PEGylation, which prevents aggregation and creates a water corona around the NP. Generally, the water corona reduces protein adsorption and opsonization, and thus prevents recognition by the reticuloendothelial system in liver and spleen. In previous studies, it has been demonstrated that the majority of opsonized particles are cleared within a few minutes due to the high concentration of phagocytic cells in the liver and spleen, or they are excreted (Alexis, et al. 2008). However, it has recently also been reported that PEG can affect the composition of the protein corona that forms around nanocarriers, and that the presence of distinct proteins is necessary to prevent non-specific cellular uptake (Schottler, et al. 2016). Different NPs used in the present invention has been demonstrated to have a circulation half-life from 45 (PBCA) to 136 min (PEBCA). Accordingly, different embodiments of the invention provide a diversity in circulation time, far enhanced compared to previous studies. The increased circulation may be due to increased PEGylation, which is achieved when PACA NPs are manufactured as described in WO 2014/191502. The NPs as used in the present invention also have a decreased degradation rate and presumably a slower dissociation/release of PEG from the particle surface. The more hydrophobic polymer (PEBCA vs PBCA) could also give a stronger anchoring of the PEG, which is attached by hydrophobic interactions. Similar half-lives in the order of a few hours have been reported also by others, for PBCA NPs loaded with doxorubicin (Reddy and Murthy 2004) and for hexadecyl cyanoacrylate (PHDCA) NPs (Fang, et al. 2006).

Further, the NPs must extravagate from the vasculature, penetrate the extracellular matrix (ECM), and deliver their payload to the intracellular targets. Several advantages have been demonstrated for the NPs to be used according to the invention. Leaky tumor vasculature and nonfunctional lymphatics result in the enhanced permeability and retention (EPR) effect, which allows the NPs to selectively extravasate and accumulate in tumors, while the healthy tissue is less exposed.

Biodistribution of NPs were demonstrated in an animal model, wherein the mice were injected intravenously with NPs containing dye. The amount of NPs accumulating in the tumor was measured when the NPs were nearly cleared from the circulation (6 h post injection), and 1% of the injected NP dose was found to be located in the tumor. This is a clear improvement compared to what has been reported for chemotherapeutic drugs, where only 0.01 to 0.001% of the injected drug reaches the tumor (Gerber, et al. 2009, Kurdziel, et al. 2011). The majority of the NPs was found in the liver and spleen, while less NPs were localized in the kidneys. This demonstrates that the NPs do not degrade much during this time period.

Cellular uptake of NPs was determined by using CLSM and flow cytometry. The model used for determining uptake utilized breast cancer cells (MDA-MB-231) and NPs encapsulating fluorescent dye. CLSM images confirmed florescent dye within the cells. In one experiment with PEBCA loaded with fluorescent dye, quantification by FCM revealed that 90% of the cells had taken up NPs by endocytosis after 3 hours.

The uptake of PACA NPs has been observed to vary between different cell lines and for NPs of different polymers. The efficient in vitro uptake of the PEBCA NPs observed for the MDA-MB-231 breast cancer cell line, indicates that once the NPs have reached the tumor interstitium, they can effectively be taken up by the breast cancer cells by endocytosis. Once the NPs have been internalized, they will degrade in order to release the cytostatic cargo. In vitro toxicity with cabazitaxel as a drug confirms that cell line responds well to the drug, and the encapsulated drug is efficient. If the NPs were not internalized, alternative mechanisms would be that the NPs degrade and release the drug extracellularly, followed by cellular uptake of the free drug, or that the drug is delivered by direct contact-mediated transfer into cells, which has been observed for another hydrophobic model drug. The degradation of PACA nanoparticles has been characterized, and occurs mainly by surface erosion after hydrolysis of the ester bond of the alkyl side chain of the polymer, resulting in degradation products of alkyl alcohol and poly(cyanoacrylic acid), which are excreted by the kidneys.

Studies have also been conducted to demonstrate the in vivo circulation of MBs, in particular the described MBs associated with NPs as a shell on the surface. With the use of an animal model, NP associated MBs were injected intravenously in mice. Biodistribution was demonstrated by contrast enhancement in a tumor imaged by US.

The MBs were injected intravenously, and could be imaged both in venous and arterial circulation using a pre-clinical US scanner. In the tumor tissue, NP-stabilized MBs could be detected for approximately 4-5 min, which is comparable to other commercial MBs.

Microdistribution of NPs in tumors was also investigated by CLSM imaging, and demonstrated that various MI influenced the microdistribution of NPs in the tumor. The result demonstrated that an increased delivery of NPs is observed in the tumors treated with US compared to the control tumor where no US is used. To determine the optimal treatment of the animals included in the model for the delivery system of the invention, and to achieve enhanced delivery of NPs in to the tumor tissue, various US treatments were investigated. Understanding the cavitation processes is crucial to maximize efficiency and safety in US-mediated drug delivery. The response of a MB to US depends highly on the frequency, pressure level and pulse duration, as well as properties of the MB such as size, shell thickness and stiffness. The effect of US-mediated delivery of NPs also depends on tumor characteristics as the barriers for delivery of nanomedicine can vary greatly between tumor types.

In the subcutaneous breast cancer model described in example 7, lower acoustic pressures (MI of 0.1 or 0.25) did not enhance tumor uptake of PEBCA NPs. Acoustic characterization and in vitro US contrast imaging of NP-stabilized MBs have shown that the NP-stabilized MBs are acoustically active and oscillate at these pressure levels, and that there is partial destruction at an MI of 0.25. Still, these low pressures did not affect the vascular permeability enough to allow extravasation of NPs in vivo in the model as described in Example 9. Delivery of larger agents such as NPs may require higher US pressures compared to delivery of low molecular weight drugs, accordingly US intensities can be adapted to create pore sizes which correlate with drug size.

At higher acoustic pressure (MI of 0.5 and 1) the delivery of NPs to tumors in the breast cancer model described herein was improved. Without being bound by theory, this may indicate that complete destruction of the NP-stabilized MB is necessary for enhanced permeability. At an MI of 0.5, there was a significantly improved tumor accumulation; the number of NPs delivered was in average 2.3 times higher than the non-treated group. If the MB is located close enough to the capillary wall, the oscillating and collapsing MB will induce forces on the endothelial cells through shear stresses, fluid streaming, shock waves and jet streams. The increased extravasation and distribution of NPs are thus likely due to one or a combination of the following; increased vascular permeability through increased number of fenestrations, increased endocytosis/exocytosis of NPs in endothelial cells, or increased fluid convection in the vasculature and interstitium. The variation in NP accumulation within treatment groups is likely due to different amount of vasculature between different tumors, as well as variations in leakiness of the vasculature, and different size of the necrotic core. In Example 9, a short flash of MI 1 did not improve the uptake of NPs, demonstrating that a longer pulse is needed. The longer pulse might push the MB towards the vessel wall, possibly resulting in a closer proximity to the endothelial cells at the time of the burst of the MB. During the long pulse, the NP-stabilized MB will burst, and the released gas can form new and possibly smaller MBs, which again will oscillate and potentially coalesce. Altogether, as demonstrated herein long pulses facilitate sustained bioeffects from the oscillating bubbles.

The direct association between the NPs and MB will probably result in a higher local concentration of NPs when the MBs are destroyed, compared to co-injection of NPs and MBs. Accordingly, the invention represents a more efficient delivery compared to a co-injection of NPs and MBs.

The invention is illustrated by the following non-limiting examples.

### EXAMPLES

### Example 1

Production of Drug-Loaded PACA NPs and NP-Stabilized Microbubbles

Materials and Methods:

Synthesis and Physico-Chemical Characterization of Drug Loaded PACA NPs:

PEG-coated and cabazitaxel-loaded PIHCA NPs were prepared by the miniemulsion method as follows: An oil phase containing 1.50 g of isohexyl cyanoacrylate (monomer), 0.03 g of Miglyol 812 (co-stabilizer, inactive oil) and 0.18 g cabazitaxel (cytotoxic drug) was prepared by thorough mixing in a glass vial. An aqueous phase containing 0.09 g of Brij L23 (23 PEG units, MW 1225) and 0.09 g of Kolliphor HS15 (15 PEG units, MW 960), dissolved in 12 ml of 0.1 M HCl was prepared. An oil-in-water emulsion was prepared by mixing the oil and aqueous phase and immediately sonicating the mixture (Branson digital sonifier 450) on ice for 2 minutes (4×30 sec intervals, 60% amplitude) followed by another 3 minutes (6×30 sec intervals, 30% amplitude). After sonication the solution was rotated at 15 rpm overnight at room temperature before adjusting the pH to 5 using 0.1M NaOH. The polymerization was continued for 5 hours at room temperature while rotated (15 rpm). The dispersion was dialyzed extensively against 1mM HCl (pH 3) at room temperature to remove unreacted PEG (dialysis membrane, MWCO 100,000 Da). The dialysate was replaced 3 times. The particles were stored in the acidic solution at 4° C. The above-mentioned method resulted in PEGylated, drug-loaded and non-targeted NP dispersions with concentrations of 75 mg NP/ml after dialysis. When stored in acidic condition, the particle dispersion was stable for several months, with no aggregation observed.

Zetasizer (Dynamic light scattering) was used in order to determine hydrodynamic size, size distribution and surface charge of the PACA nanoparticles. To calculate the amount of encapsulated drug, drug content was extracted from the particles and the extracted amount of cabazitaxel was quantified by using LC-MS/MS method.

Production and Characterization of NP-Stabilized MBs:

Gas-filled MBs associated with PACA NPs were produced as follows: A solution containing 2 wt % casein (pH 7) was prepared and filtered through 0.22 μm syringe filter. The cabazitaxel-loaded PEGylated PIHCA NPs described above were mixed with the casein solution and distilled water to a final concentration of 0.5 wt % casein and 1 wt % NP, with a total volume of 4 ml. The mixture was placed in a sonication batch for 10 minutes (at ambient temperatures) before the solution was saturated with perfluoropropane gas (approximately 10 seconds) and the vial partly sealed with parafilm. Ultraturrax (25,000 rpm) was then immediately applied for 2 minutes to produce perfluoropropane-filled NP-stabilized MBs. The vial was immediately sealed under perfluoropropane atmosphere using septum.

The size and concentration of the resulting NP-stabilized MBs was determined from light microscopy images using a 20× phase contrast objective and cell counter. MBs were counted and the size was calculated by analyzing the images.

Results:

The above-mentioned method resulted in PEGylated, drug-loaded and non-targeted NP dispersions with concentrations of 75 mg NP/ml after dialysis. When stored in acidic condition, the particle dispersion was stable for several months, with no aggregation observed.

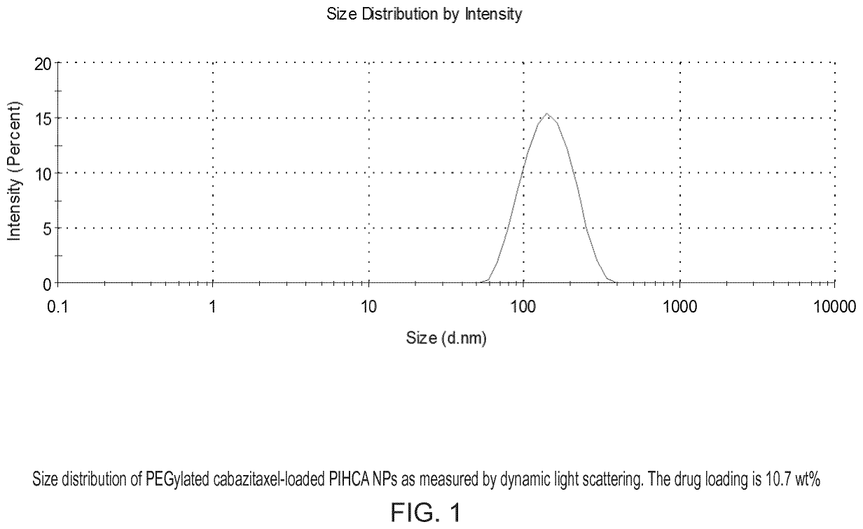

Dynamic light scattering method showed an NP size of 142 nm (z-average) with a polydispersity index of 0.18 (see FIG. 1). The measured zetapotential was −1 mV. The determined drug loading efficiency was 72% and the drug payload was 10.7% (% wt cabazitaxel/wt NP).

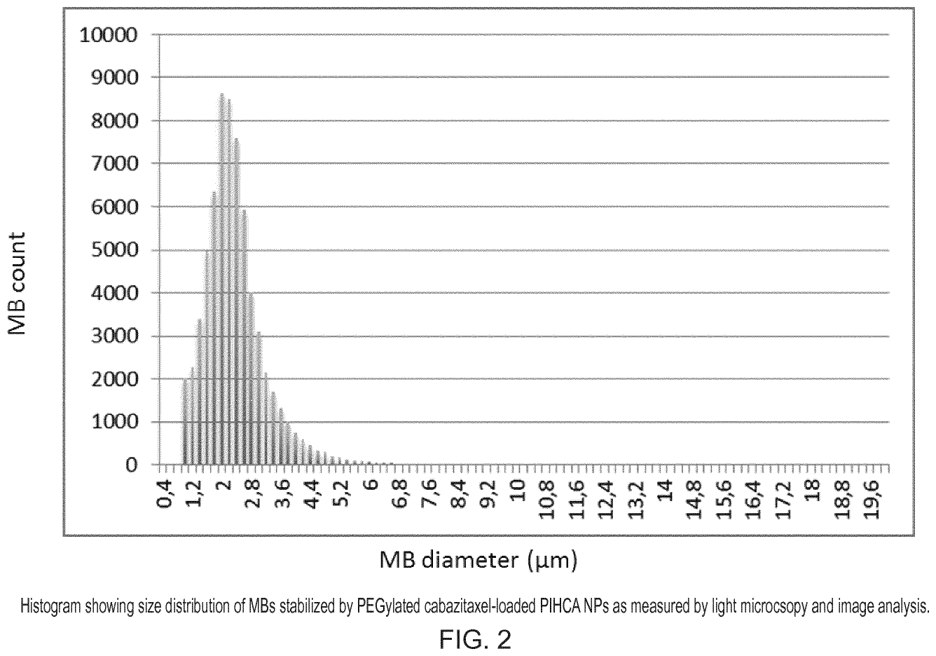

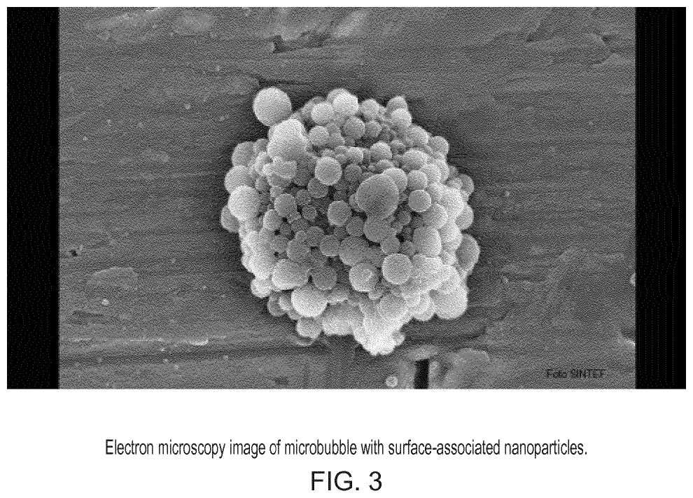

The resulting NP-stabilized MBs had an average size of 2.3 μm (see FIG. 2) and concentration of 5.62E+08 MBs/m1 as measured by light microscopy and image analysis. Fluorescence microscopy (using same type of NPs only encapsulating a fluorescent dye instead of drug) and electron microscopy (FIG. 3) was used to confirm that NPs are associated with the MBs forming a stabilizing (mono)layer. When stored at 4° C., the microbubbles were stable for up to several months.

### Example 2

Cellular uptake of fluorescent dye (“model drug”) encapsulated in nanoparticles (PIHCA) in breast cancer cells.

The aim of this study was to investigate the mechanisms of ultrasound-mediated delivery, to determine whether stable or inertial cavitation is the major mechanism for improved extravasation and enhanced NP delivery. To achieve successful delivery, the NPs have to circulate in blood for sufficient amount of time, extravasate from the vasculature, penetrate the extracellular matrix and deliver their payload to the intracellular targets.

Size and zetapotential of the biocompatible and biodegradable poly(isohexyl cyanoacrylate) NPs were determined by Zetasizer. In vitro cellular uptake was studied in breast cancer cells (MDA-MB-231) using confocal laser scanning microscopy (CLSM) and flow cytometry (FCM) by encapsulating a fluorescent dye.

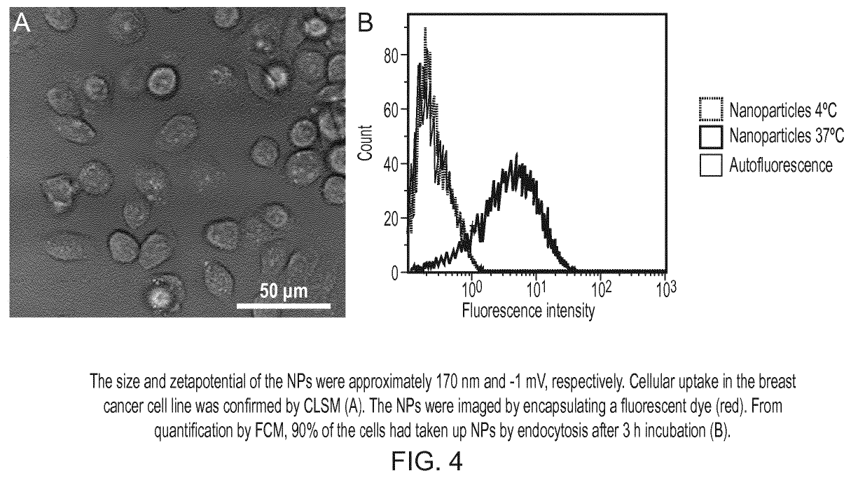

FIG. 4 shows that the size and zetapotential of the NPs were approximately 170 nm and −1 mV, respectively. Cellular uptake in the breast cancer cell line was confirmed by CLSM (see A). The NPs were imaged by encapsulating a fluorescent dye. From quantification by FCM, 90% of the cells had taken up NPs by endocytosis after 3 h incubation (B).

In vivo circulation half-life of NPs was determined by blood sampling from the saphenous vein in mice at 10 min, 30 min, and 1, 2, 4, 6, and 24 h post injection.

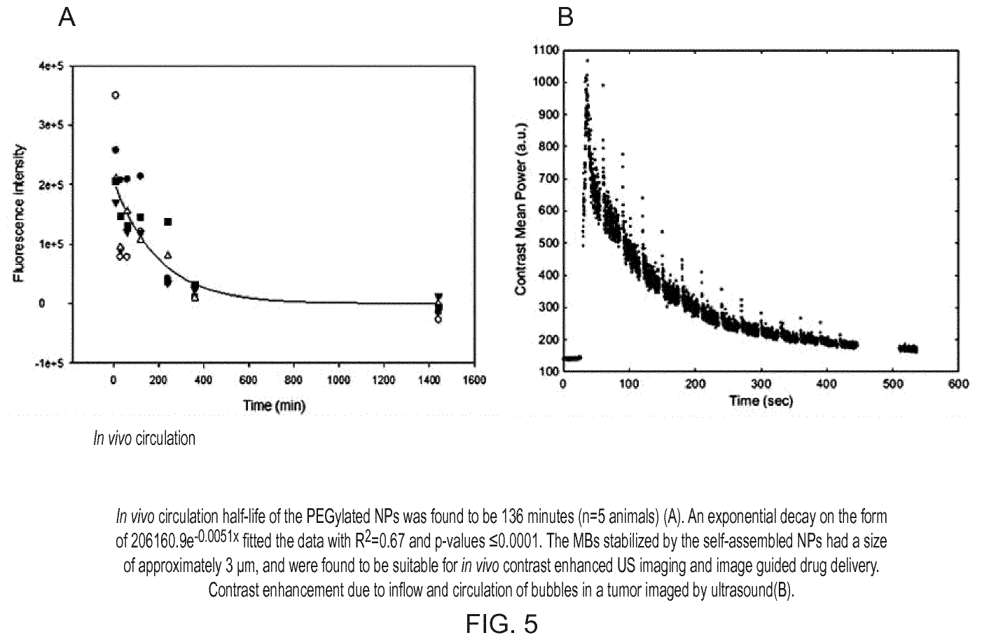

FIG. 5 shows in vivo circulation half-life of the PEGylated NPs. It was found to be 136 minutes (n=5 animals) (A). An exponential decay on the form of 206160.9e^{-0.0051x} fitted the data with R²=0.67 and p-values ≤0.0001. The MBs stabilized by the self-assembled NPs had a size of approximately 3 μm, and were found to be suitable for in vivo contrast enhanced US imaging and image guided drug delivery. Contrast enhancement due to inflow and circulation of bubbles in a tumor imaged by ultrasound (see FIG. 5, B).

Perfluoropropane MBs were made by vigorous stirring and self-assembly of the NPs at the gas-water interface. Inflow and circulation of microbubbles in tumors was imaged by ultrasound at 18 MHz.

Biodistribution of NPs encapsulating a near infrared dye was imaged 6 h post injection.

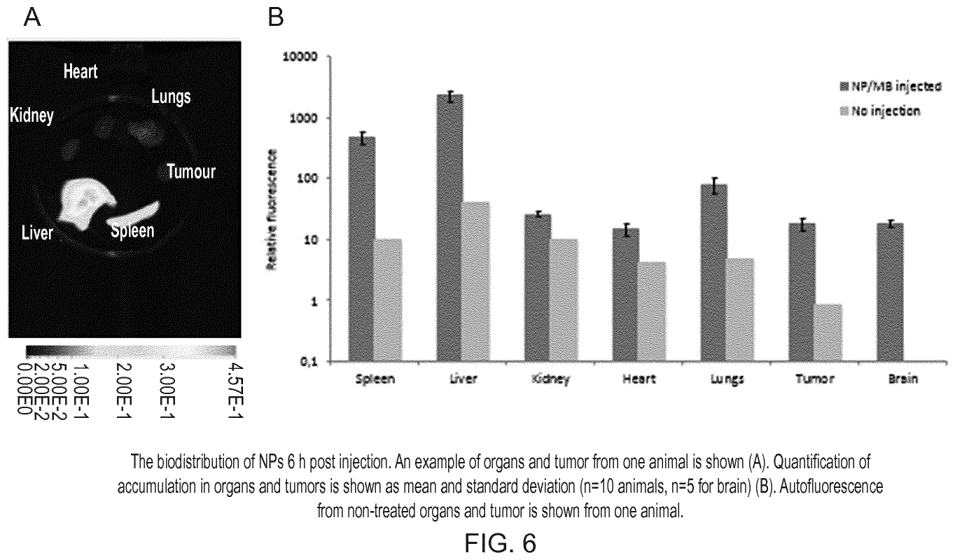

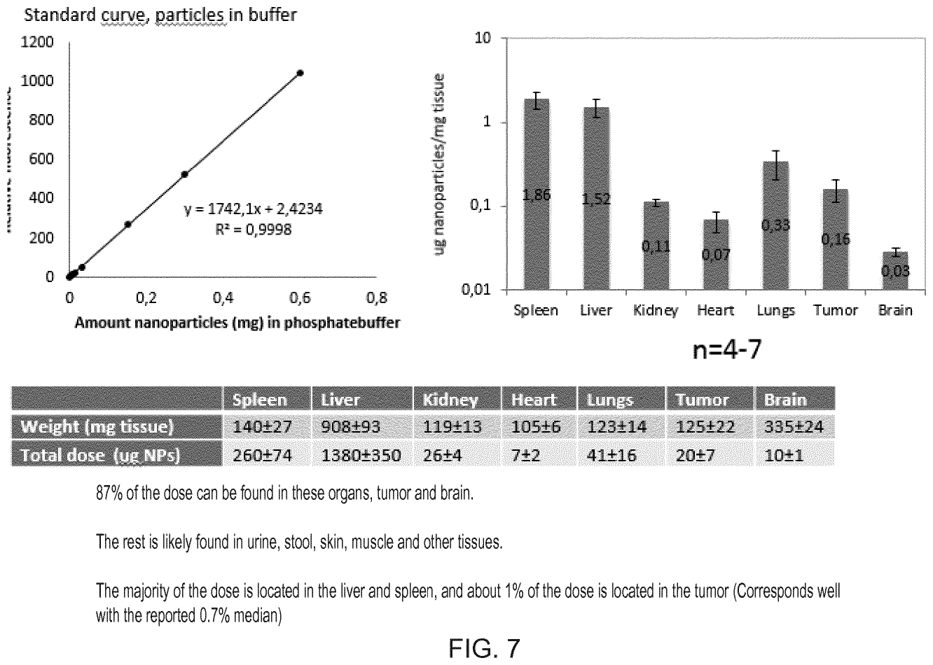

The biodistribution of NPs was determined by imaging using a near infrared whole animal scanner, and by ex vivo quantification of accumulation in excised organs and tumors. This is presented in FIGS. 6 and 7.

FIG. 6 shows the biodistribution of NPs 6 h post injection. An example of organs and tumor from one animal is shown (A). Quantification of accumulation in organs and tumors is shown as mean and standard deviation (n=10 animals, n=5 for brain) (B). Autofluorescence from non-treated organs and tumor is shown from one animal.

FIG. 7 shows that 87% of the dose can be found in these organs, tumor and brain. The rest is likely found in urine, stool, skin, muscle and other tissues. The majority of the dose is located in the liver and spleen, and about 1% of the dose is located in the tumor (Corresponds well with the reported 0.7% median)

To study how stable versus inertial cavitation of MBs affected NP uptake in tumor tissue, subcutaneous breast cancer xenografts (MDA-MB-231) were grown in athymic mice. When tumors reached 7-8 mm length, MBs stabilized by NPs were injected intravenously before the tumors were treated with one of six different FUS treatments, using a 1 MHz FUS transducer and MIs ranging from 0.1 to 1. Blood vessels were stained by injecting FITC-labeled tomato lectin. The microdistribution of NPs was imaged by CLSM on frozen tumor sections. The experimental setup and the different treatment groups are indicated below:
G1: Control group, no ultrasound.
G2: 0.5 sec treatment, 1.5 sek break, (global PRF=0.5 Hz), 10.000 cycles (10 ms) every 100 ms, (local PRF=10 Hz), total duty cycle 2.5%, MI 0.1 (A).
G3: As G2 with 3 additional cycles flash of MI 1 after each treatment (B).
G4: As G3, but only the flash of MI 1 (C) .
G5: As G2 but with an MI 0.25.
G6: As G2 but with an MI 0.5 (D).
G7: As G2 but with an MI 1.

Results

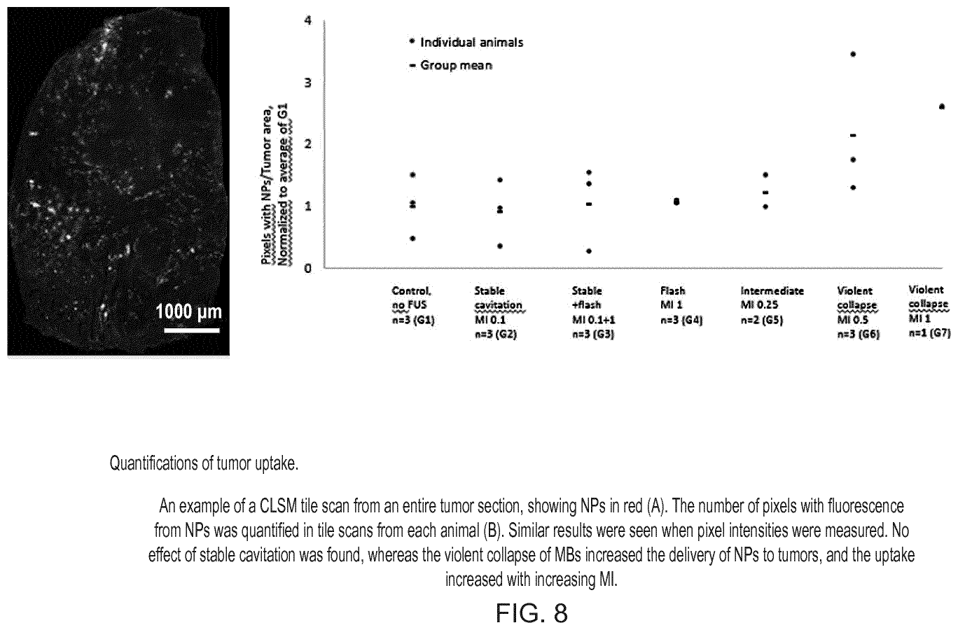

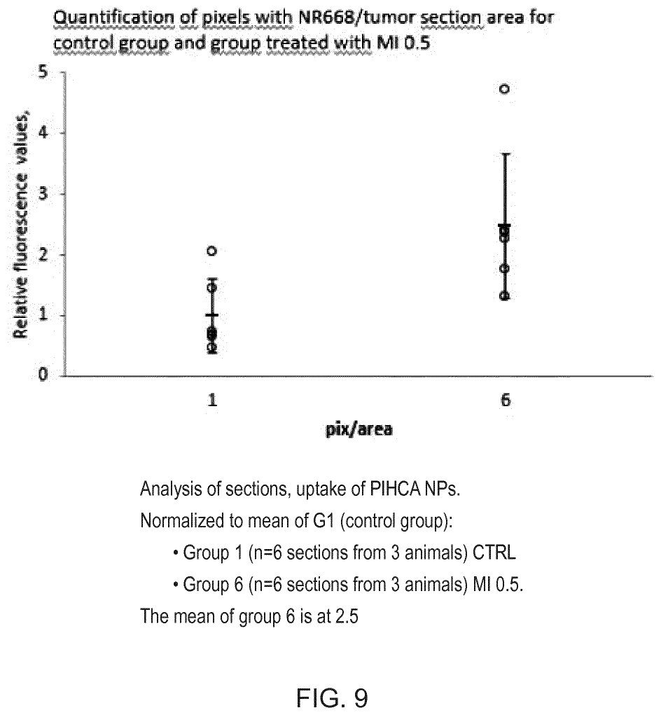

Results are presented in FIGS. 8, 9 10 and 11.

FIG. 8 demonstrate an example of a CLSM tile scan from an entire tumor section, showing NPs in red (A). The number of pixels with fluorescence from NPs was quantified in tile scans from each animal (B). Similar results were seen when pixel intensities were measured. No effect of stable cavitation was found, whereas the violent collapse of MBs increased the delivery of NPs to tumors, and the uptake increased with increasing MI.

FIG. 9 shows analysis of sections and uptake of PIHCA NPs. The results of G1 is compared to G6.

Normalized to mean of G1 (control group):
Group 1 (n=6 sections from 3 animals) CTRL
Group 6 (n=6 sections from 3animals) MI 0.5.

The mean of group 6 is at 2.5

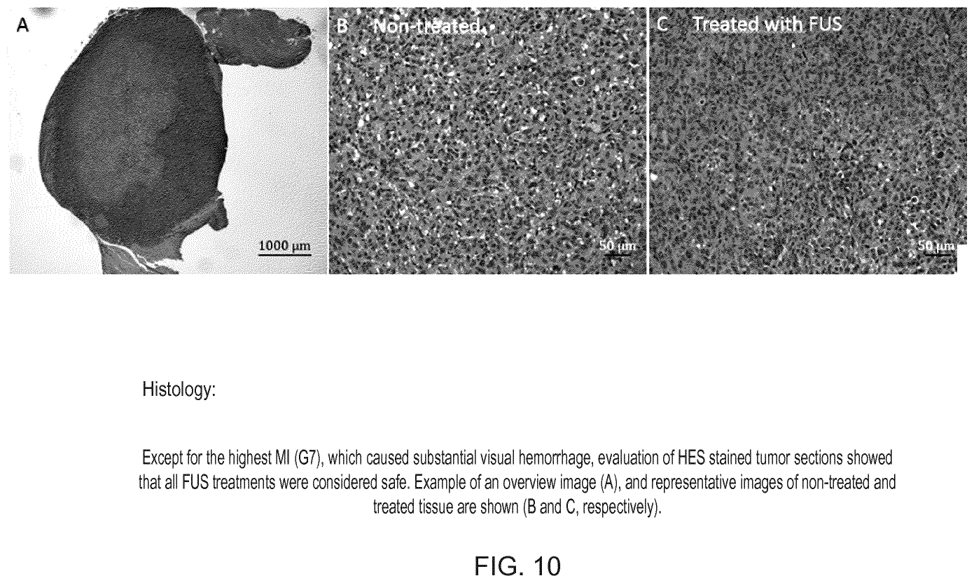

Hematoxylin erythrosine saffron (HES) stained sections were imaged to evaluate safety of the treatment. FIG. 10 shows the evaluation of the safety analysis. Except for the highest MI (G7), which caused substantial visual hemorrhage was analyzed. The evaluation of HES stained tumor sections showed that all FUS treatments were considered safe. Example of an overview image (A), and representative images of non-treated and treated tissue are shown (B and C, respectively).

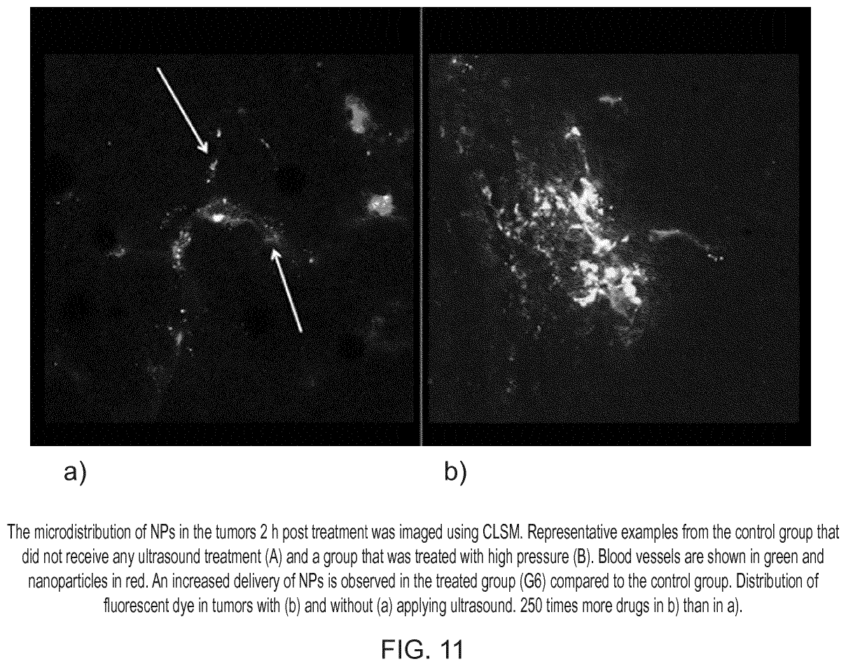

The micro distribution of NPs was imaged on frozen tumor sections using confocal laser scanning microscopy. This is presented in FIG. 11, which shows the microdistribution of NPs in the tumors 2 h post treatment as imaged using CLSM. Representative examples from the control group that did not receive any ultrasound treatment (A) and a group that was treated with high pressure (B). Blood vessels are shown in green and nanoparticles in red. An increased delivery of NPs is observed in the treated group (G6) compared to the control group. Distribution of fluorescent dye in tumors with (b) and without (a) applying ultrasound. The image show approximately 250 times more drugs in b) with the use of ultrasound than in a) without ultrasound.

Conclusion

High pressure sonication and thus violent collapse of MBs was found to improve the delivery of NPs to tumors, and increasing uptake was observed with increasing MI.

However, hemorrhage was observed at the highest MI used, indicating that high MI in combination with MBs should be used with caution for drug delivery purposes.

The results show that this NP-MB platform is highly useful for controlled drug delivery.

### Example 3

Uptake of drug in cells and cytotoxicity of empty and drug-loaded PACA NPs

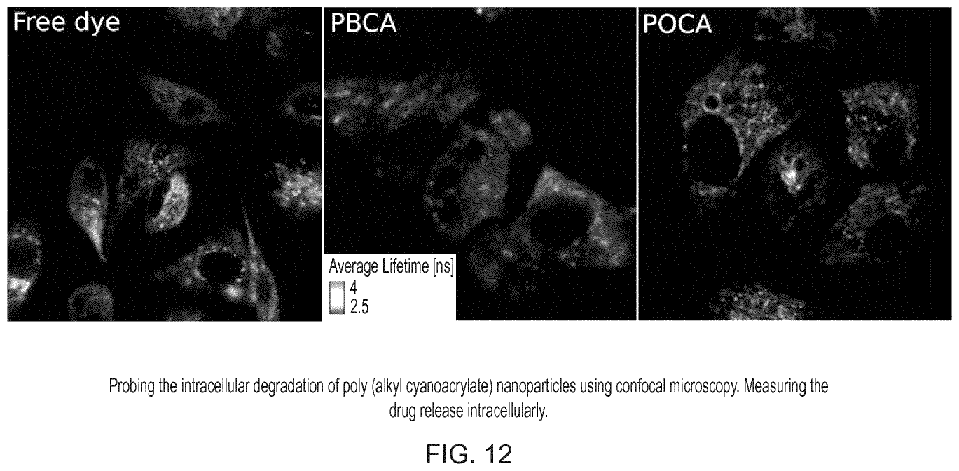

Measuring the drug release intracellularly is necessary in order to understand the effect on cancer cells after internalization. The inventors used the model drug NR668 (modified Nile Red) encapsulated in poly (butyl cyanoacrylate) (PBCA) and poly (octyl cyanoacrylate) (POCA) to demonstrate that the NPs have different drug release kinetics also after internalization. While ordinary fluorescence imaging gives little information about the degradation, Fluorescence lifetime imaging (FLIM) (as shown in FIG. 12), Förster resonance energy transfer (FRET), emission specter analysis and time-laps imaging after cell lysis provids valuable information.

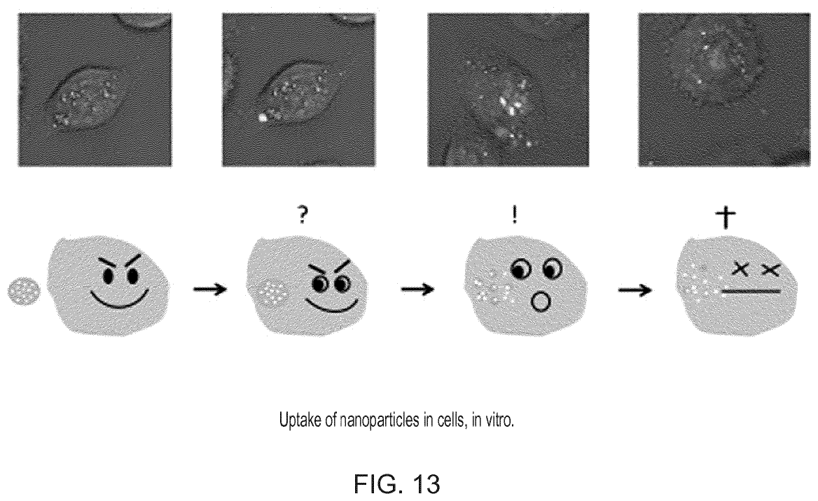

FIG. 13 demonstrate the cellular uptake of NPs in breast cancer cells.

The cytotoxic effect of empty PBCA NPs, PBCA NPs with encapsulated cabazitaxel as well as free cabazitaxel was studied on breast cancer cells (MDA-MB-231 cells=human epithelial, mammary adenocarcinoma cell line). AlamarBlueR Cell Viability Assay was used to evaluate cell viability. Cells were seeded in density 5000 cells/200 μl medium for each well. After 3 days old medium was removed from wells and both encapsulated cabazitaxel and free cabazitaxel was diluted in medium and added to the well. Concentration of NPs was ranged from 0.1 ng/ml to 1000 ng/ml. Concentrations of free cabazitaxel was chosen to match the concentrations of cabazitaxel in NPs. Control wells contained cells in growth medium. The particle size was approximately 125 nm for empty NPs and approximately 160 nm for both drug-loaded NPs.

The well plates were incubated for 24, 48 and 72 hours at 37° C. and 5% CO₂, before the medium was removed from the well followed by 3 times washing with fresh growth medium. Growth medium containing 10% of alamar Blue assay was added into each well and the plates incubated for another 3 hours at 37° C. and 5% CO₂, and the fluorescence intensity measured by microplate reader (excitation/emission at 550/590 nm).

Results:

The MDA-MB-231 cells responded to treatment with encapsulated cabazitaxel in PBCA and free cabazitaxel at various concentrations in a dose-responsive manner (FIG. 14). The cytotoxic effect of encapsulated cabazitaxel was similar to free cabazitaxel, demonstrating the successful release of drug from the particles.

Similar effects were seen with other PACA NPs (PIHCA and POCA) and with other cell lines (P3 glioma and HeLa cells).

### Example 4

FUS-Mediated BBB Opening

Methods

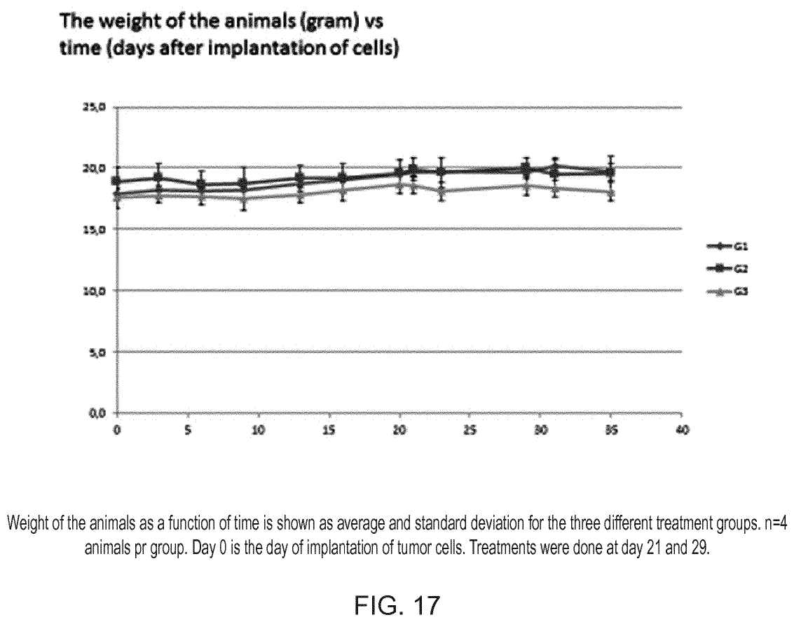

For FUS-mediated BBB opening, the inventors used a state-of-the-art ultrasound system able to generate FUS at 1.1 MHz and 7.8 MHz during the same experiment, allowing a very precise magnetic resonance imaging (MRI)-guided selection of the area exposed to FUS. FUS exposure at the lower frequency was used to disrupt the BBB. FUS at the higher frequency of 7.8 MHz was employed to enable the effect of the acoustic radiation force. This force is caused by a transfer of momentum between the ultrasound wave and the propagation tissue, and the hypothesis is that it can facilitate NP transport in the extracellular matrix. Experiments were performed on immunodeficient mice with melanoma brain metastases developed four weeks after intracardiac injection of patient-derived human melanoma cells. A NP-MB platform, based on PIHCA NPs forming a shell around perfluorocarbon MBs, was used for FUS-mediated BBB opening. PIHCA NP-MBs were injected immediately before the FUS exposure. BBB opening was assessed using a gadolinium-based contrast agent. After the experiments, the brains were either frozen or fixed in formalin. NP transport across the BBB and distribution in the brain tissue were assessed in cryosections using confocal microscopy (see FIG. 17) , while histopathological changes and cellular changes caused by FUS were evaluated using formalin-fixed paraffin embedded tissue sections.

Results and Conclusions

FIG. 15 shows uptake of the MRI contrast agent dye in brain. This specific agent will normally not pass the BBB. Thus, the results illustrate transient BBB opening

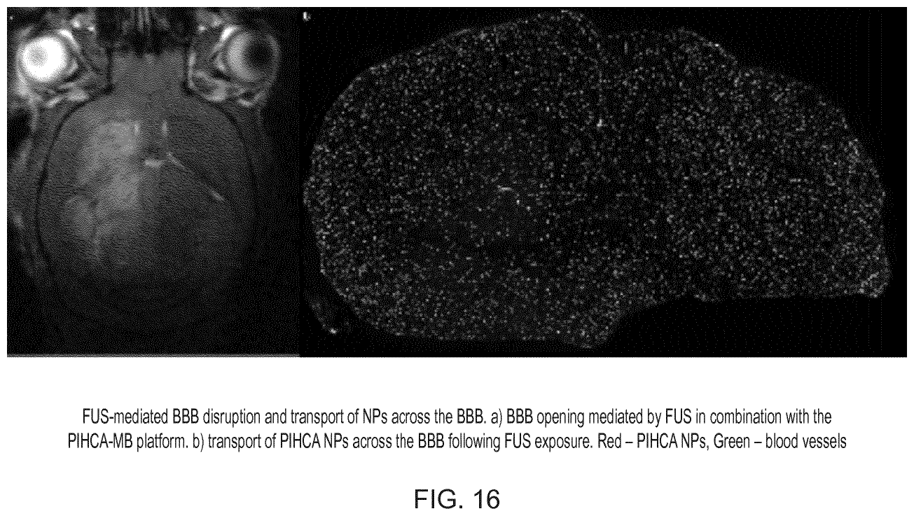

FIG. 16 demonstrate FUS-mediated BBB disruption and transport of NPs across the BBB. In a) one can see BBB opening mediated by FUS in combination with the PIHCA-MB platform. In b), transport of PIHCA NPs across the BBB following FUS exposure. Red—PIHCA NPs, Green—blood vessels

Successful BBB opening was verified by MRI (as shown in FIG. 15). An optimal window for FUS-mediated BBB disruption using our NP-MB platform was found to be around a mechanical index of 0.31. Analysis of cryosections showed that the combination of FUS with our NP-MB platform allowed transport of NPs across the BBB in an ÷opening-dependent manner. Histological evaluation showed some extent of red blood cell extravasation following FUS exposure. The effect of the acoustic radiation force of NP distribution in the brain parenchyma away from blood vessels and the effect of FUS exposure on P-glycoprotein, an efflux transporter that is an integral part of the BBB, are currently being analysed. Overall, our results indicate that our platform based on PIHCA NPs and MBs can be used to deliver substantial amount of NPs across the BBB, showing its potential in NP-aided drug delivery to the brain.

### Example 5

In Vivo Demonstration of Therapeutic Effects

In vivo studies of effect of ultrasound-mediated drug delivery of MBs associated with NP loaded with anti-cancer drug in treatment of tumors.

The aim of the study was to investigate the described drug delivery systems ability to treat cancer, i.e. stop abnormal cell growth and shrinkage of tumors, in an in vivo model. The cancer cell used to demonstrate the potential of the invention was breast cancer cells, and the therapeutic agent was cabazitaxel.
MDA-MB-231 breast cancer cells implanted subcutaneously on nude mice on day 0
Tumors were allowed to grow until they reached a diameter of 4 mm in the longest direction (some just above and some just below 4)
4 animals were included in each group. Group 1: saline. Group 2: microbubbles associated with NPs loaded with cabazitaxel. Group 3: Microbubbles associated with NPs loaded with cabazitaxel and ultrasound.
Injected volume was 200 ul intraveneously, total 2 mg nanoparticles per animal, and approximately 10 mg/kg cabazitaxel
Ultrasound treatment was optimized previously, and an MI of 0.5 was used.
The mice were treated on day 21 and day 29
Because the imasonic 1 MHz transducer stopped working, the second ultrasound treatment had to be done with the FUS equipment. 16 spots (4×4) were scanned to cover the tumor area. The transducer had to be scanned because of the small focus. In each spot, 10000 cycles were given, and the 16 spots were scanned during 3.5 seconds. Total treatment time was increased from 2 minutes with the previous imasonic, to 3.5 minutes with the FUS equipment.
Tumor growth is measured using calipers

The results of the study are presented in FIG. 17-19.

Conclusion

The study demonstrates enhanced delivery of therapeutic agent to tumors, and show a therapeutic effect of the drug delivery system according to the invention.

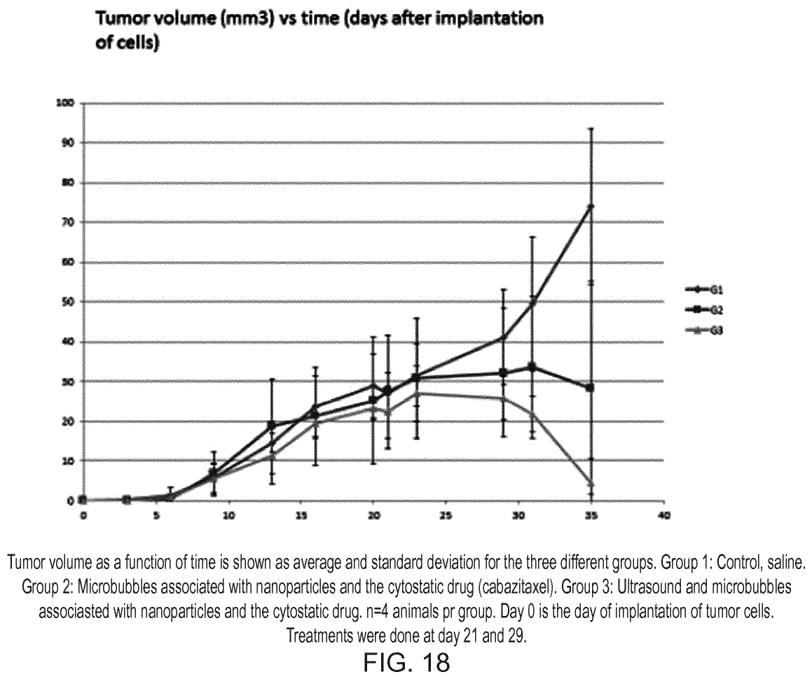

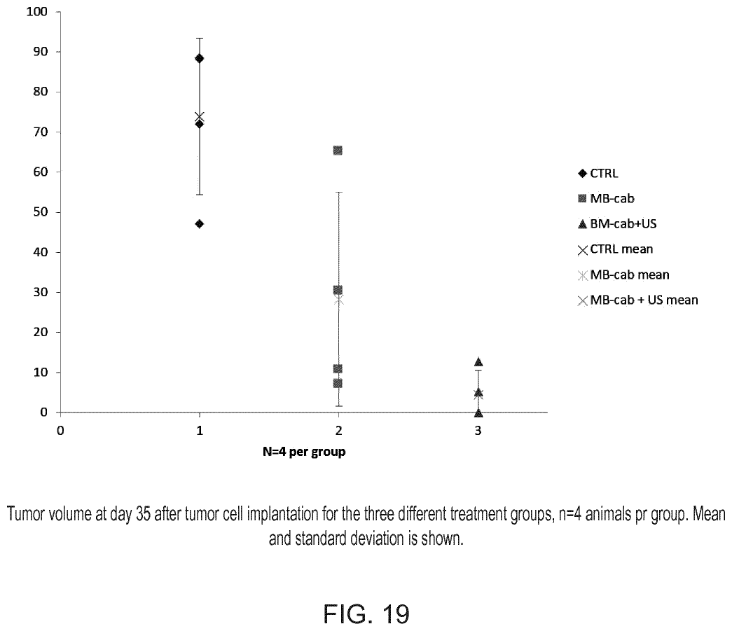

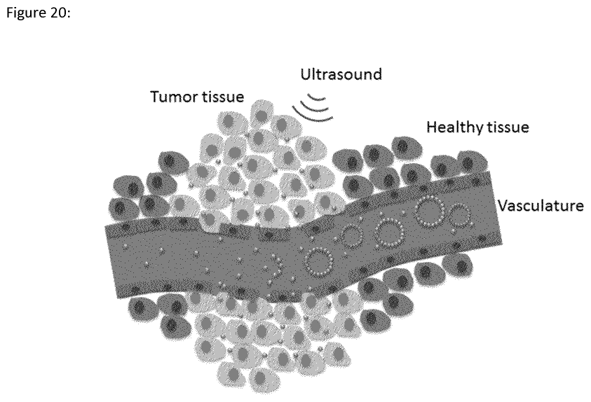

The tumors in the control group (saline) grow at a certain rate, illustrated with the upper (=blue) curve in FIG. 18. Animals that are treated with microbubbles containing nanoparticles and the cytostatic drug (cabazitaxel) show reduced tumor growth (the curve in the middle=red curve). Animals which are treated with ultrasound in addition to microbubbles and nanoparticles filled with the cytostatic drug show that the tumor growth stops, the tumors shrink, and 2 out of 4 animals are cured at this time point (the lower curve =green curve). FIGS. 18 and 19 shows the effect achieved with the treatment. The weight of the animals was stable during and after the treatment for all three groups (see FIG. 17), proving that the treatment was well tolerated.

FIG. 17: Weight of the animals as a function of time is shown as average and standard deviation for the three different treatment groups. n=4 animals pr group. Day 0 is the day of implantation of tumor cells. Treatments were done at day 21 and 29.

FIG. 18: Tumor volume as a function of time is shown as average and standard deviation for the three different groups. Group 1: Control, saline. Group 2: Microbubbles associated with nanoparticles and the cytostatic drug (cabazitaxel). Group 3: Ultrasound and microbubbles associasted with nanoparticles and the cytostatic drug. n=4 animals pr group. Day 0 is the day of implantation of tumor cells. Treatments were done at day 21 and 29

FIG. 19: Tumor volume at day 35 after tumor cell implantation for the three different treatment groups, n=4 animals pr group. Mean and standard deviation is shown

### Example 6

### Production of Drug-Loaded PEBCA NPs and PEBCA-Stabilized Microbubbles

Synthesis and Characterization of Nanoparticles and Microbubbles

PEGylated PEBCA NPs were synthesized by miniemulsion polymerization as described previously (Mørch, et al. 2015). Briefly, an oil phase consisting of 2-ethyl-butyl cyanoacrylate (monomer, Henkel Loctite, Dusseldorf, Germany) containing 0.1 wt % methane sulfonic acid (Sigma-Aldrich, St. Louis, Mo., USA), 2 wt % Miglyol 812 (co-stabilizer, Cremer, Cincinnati, Ohio, USA) and 0.8 wt % azo bis-dimethyl valeronitril (V65, oil-soluble radical initiator, Waco, Osaka, Japan) was prepared. Fluorescent particles for optical imaging were prepared by adding either NR668 (modified NileRed (Klymchenko, et al. 2012), custom synthesis, 0.5 wt %) or IR-780 Lipid (near-infrared dye, custom synthesis, CEA, Grenoble, France, 0.5 wt %) to the oil phase. Particles containing cytostatic drug for treatment were prepared by adding cabazitaxel (10 wt %, Biochempartner, Wuhan, Hubei, China) to the oil phase.

An aqueous phase consisting of 0.1 M HCl containing Brij L23 (10 mM, 23 PEG units, MW 1225, Sigma-Aldrich) and Kolliphor HS15 (10 mM,15 PEG units, MW 960, Sigma-Aldrich) was added to the oil phase and immediately sonicated for 3 min on ice (6×30 sec intervals, 60% amplitude, Branson Ultrasonics digital sonifier 450, Danbury, Conn., USA). The solution was kept on magnetic stirring for 1 h at room temperature before adjusting the pH to 5 using 0.1M NaOH. The polymerization was continued for 2 h at room temperature before increasing the temperature to 50° C. for 8 h while the solution was rotated (15 rpm). The dispersion was dialyzed (Spectra/Por dialysis membrane MWCO 100,000 Da, Spectrum Labs, Rancho Dominguez, Calif., USA) against 1 mM HCl to remove unreacted PEG. The dialysate was replaced 3 times. Details regarding PEGylation of NP-platform have been published previously (Baghirov, et al. 2017, Mørch, et al. 2015, Åslund, et al. 2017). The size, polydispersity index (PDI) and the zeta potential of the NPs were measured by dynamic light scattering using a Zetasizer Nano Z S (Malvern Instruments, Malvern, UK). To calculate the amount of encapsulated drug, the drug was extracted from the particles by dissolving them in acetone (1:10), and quantified by liquid chromatography coupled to mass spectrometry (LC-MS/MS, Agilent 6490 triple quadrupole coupled with Agilent 1290 HPLC, Agilent Technologies, Santa Clara, Calif., USA).

NP-stabilized MBs (also referred to as NPMB) were prepared by self-assembly of the NPs (1 wt %, 10 mg/ml) at the gas-water interface by the addition of 0.5% casein in phosphate-buffered saline and vigorous stirring using an ultra-turrax (T-25, IKAWerke, Staufen, Germany) as described (Mørch, et al. 2015). Perfluoropropane (F2 Chemicals, Preston, Lancashire, UK) was used instead of air for increased circulation time. The average MB diameter, size distribution and concentration were determined using light microscopy and image analysis (ImageJ 1.48v, National Institute of Health, Bethesda, Mass., USA). The NPMB solution is a combination of free NPs and NPMBs, where only a small percentage of the NPs are located on MBs. The MBs where characterized with respect to acoustic destruction as described below (example 8).

Results:

Characterization of Nanoparticles and Microbubbles

The NPs had diameters in the range of 140-195 nm (z-average), a PDI below 0.2 and zeta-potential in the range of −1 to −2.5 mV. The determined loading efficiency of cabazitaxel was close to 100% with a drug payload of 10 wt %.

The self-assembled MBs had an average mean diameter of 2.6±1.3 μm. The concentration of MBs was approximately 5*10⁸ MBs/ml. From characterization in the in vitro flow phantom, the MBs showed no destruction at MI 0.1, partial destruction at MI 0.2 and complete destruction at MI 0.5.

### Example 7

### Treatment of Subcutaneous Xenograft Tumors

Animals and Tumors

All experimental procedures were approved by the Norwegian Animal Research Authorities. Female Balb/c nude mice (Envigo, Cambridgeshire, United Kingdom) were purchased at 7-8 weeks of age, 16-21 g. They were housed in specific pathogen free conditions, in groups of 4-5 in individually ventilated cages (Model 1284 L, Tecniplast, Lyon, France) at temperatures of 22-23° C., 50-60% relative humidity, 70 air changes per h, with ad libidum access to food and sterile water.

Subcutaneous xenograft tumors were grown from breast cancer MDA-MB-231 cells. Animals were anesthetized by inhalation of 2-3% isoflurane in O2 and NO2 (Baxter, Deerfield, Ill., USA), before 50 μl medium containing 3×10⁶ cells was slowly injected subcutaneously on the lateral aspect of the left hind leg, between the knee and the hip. During the following weeks, the animals were weighed and tumors measured using calipers 2-3 times a week. Tumor volume was calculated by π|w²/6, where 1 and w are the length and width of the tumor, respectively. Tumor growth did not affect the weight of the animals.

During experiments, the animals were anesthetized by a subcutaneous injection of fentanyl (0.05 mg/kg, Actavis Group HF, Hafnarfirdi, Iceland), medetomidine (0.5 mg/kg, Orion Pharma, Oslo, Norway), midazolam (5 mg/kg, Accord Healthcare Limited, North Harrow, United Kingdom), water (2:1:2:5) at a dose of 0.1 ml per 10 g. When necessary, a subcutaneous injection of atipemazol (2.5 mg/kg, Orion Pharma, Oslo, Norway), flumazenil (0.5 mg/kg, Fresenius Kabi, Bad Homburg vor der Hohe, Germany), water (1:1:8) at a dose of 0.1 ml per 10 g was used as antidote to terminate the anesthesia. During all experiments, the body temperature of the animals was maintained by external heating and eyes were kept moist with Viscotears Liquid gel (Alcon, Fort Worth, Tex., USA). At the end of the experiment, anesthetized animals were euthanized by cervical dislocation.

Ultrasound Setup

A custom made, single element focused transducer with a center frequency of 1 MHz (Imasonic, Besancon, France) was used. The signal was generated by a waveform generator (33500B, Agilent Technologies, Santa Clara, Calif., USA), and amplified by a 50 dB power amplifier (2100L, E&I, Rochester, N.Y., USA). The transducer was mounted at the bottom of a water chamber, and a lid with an absorber was placed at the water surface. The animals were placed on the lid, and the tumor-bearing leg lowered into the water through a 10 mm opening. The tumor was placed in the far field of the FUS beam at a distance of 190 mm, to cover the entire tumor. The water in the tank was heated to 34° C. (Trixie aqua pro heater, Zoopermarked, Hojbjerg, Denmark) to avoid hypothermia and altered blood flow in the mouse leg (Hyvelin, et al. 2013). The transducer had a diameter of 50 mm and a focal distance of 125 mm. It was characterized in a water tank using a hydrophone (HGL-0200, Onda, Sunnyvale, Calif., USA). The lateral 3 dB and 6 dB beam widths at 190 mm had diameters of 6 mm and 10 mm, respectively. In the axial direction, a 3 dB reduction in pressure was measured at 210 mm.

Characterization of Microbubble Destruction

Destruction of the NPMBs was evaluated by imaging NPMBs in an in-vitro flow phantom (model 524, ATS Laboratories, Bridgeport, Conn., USA) were the flow was driven by a peristaltic pump. The NPMBs were sonicated (1000 cycles, PRF=100 Hz) at MIs of 0.1, 0.2 and 0.5 using the 1 MHz transducer (Imasonic) while flowing through the tube of the phantom. Simultaneously, a section of the tube downstream from the sonicated region was imaged using pulse inversion at an MI of 0.07 by a clinical US scanner in contrast mode (Vivid E9 scanner and 9L transducer, GE Healthcare, Chicago, Ill., USA). Destruction of MBs was determined by visual inspection.

Ultrasound Exposure Optimization

To investigate how various acoustical settings in combination with the described MBs affected NP accumulation in tumor tissue, subcutaneous tumors in 18 mice were allowed to grow for 4-8 weeks until they had reached a diameter of approximately 7-8 mm in the longest direction and a volume of approximately 120-250 mm³. The animals were anesthetized and the lateral tail veins were cannulated, and NPMBs containing NR668 were injected intravenously, at a dose of 200 μl with 10 mg/ml NPs (100 mg/kg). The US treatment was initialized when the injection started. The mice were randomly distributed in different groups, and tumors were treated with different FUS treatments. Acoustic pressures ranged from 0.1 to 1 MPa (MIs ranging from 0.1 to 1). All tumors (except group 4) received bursts of 10 000 cycles (10 ms) every 100 ms (local PRF 10 Hz) for 0.5 s treatment, followed by 1.5 s break (global PRF 0.5 Hz, and total duty cycle 2.5%). In the groups where MB destruction was expected, reperfusion of MBs in the sonicated area was important to allow new MBs to reach the tumor, and thus a PRF of 0.5 Hz was used. For the highest pressure, a short flash of 3 cycles was also investigated. The total treatment time was 2 min.

Treatment of Triple Negative Breast Cancer MDA-MB-231 Xenografts with Nanoparticle-Microbubble Encapsulated Cabazitaxel

The tumors were allowed to grow for 3 weeks until they had reached approximately 4 mm in the longest direction. The number of animals and control groups was, in compliance with the “3Rs” (replacement, reduction, refinement)(Fenwick, et al. 2009), kept low in this pilot study. 12 animals were randomly distributed into 3 groups;
1. Animals injected with saline, control group
2. Animals injected with NPMB containing cabazitaxel
3. Animals injected with NPMB containing cabazitaxel and tumors exposed to the previously described US treatment (MI=0.5).

The mice were treated two weeks in a row (day 21 and 29 after implantation of cells). At the day of treatment, animals were anesthetized and the tail vein cannulated. An intravenous bolus of 200 μl saline or NPMB, produced as described in Example 6 was given. The concentration of NP in the bubble solution was 10 mg/ml, resulting in a total dose of 2 mg NPs per animal, and thus 10mg/kg cabazitaxel. This dose was chosen based on littératures (Semiond, et al. 2013, Vrignaud, et al. 2014, Vrignaud, et al. 2013). The optimal US treatment from the optimization of various MIs was used (the group with an MI of 0.5 as described in Example 8) for the first treatment. The second treatment was done with another transducer (RK-100 system, aperture 52 mm and focal distance 60 mm, FUS Instruments, Toronto, ON, Canada) with a frequency of 1.1 MHz. Due to a smaller focal diameter, the transducer was scanned to cover the tumor area. 16 spots (4×4) were scanned during 3.5 sec. In each spot, a burst of 10 000 cycles was transmitted. The total treatment of the second treatment time was increased from 2 min, to 3.5 min to achieve 60 sonications, to make the treatment as similar as possible to that of the first treatment with the Imasonic transducer. The lateral 3 dB and 6 dB beam widths were 1.3 and 1.6 mm, respectively, while in the axial direction, 4 cm has a pressure within the 3 dB limit.

After the treatment, the antidote was administered to terminate anesthesia, and the animals were placed in a recovery rack until the next morning to avoid hypothermia in the recovery period. The rack kept a temperature of 28° C. The days following a treatment, the animals were given Diet gel boost (ClearH2O, Westbrook, Me., USA) as a supplement to the dry food. The tumor growth was measured using calipers and the animals were weighed 2 times per week for 14 weeks after end of treatment. The criteria for humane endpoints where animals were euthanized were tumor size of 15 mm diameter or weight loss of 15%.

Statistical Analysis

A two-tailed unpaired t-test was used to evaluate if the difference in NP uptake between group 1 and 6 was statistically significant (Excel 2010, Microsoft, Redmond, Wash., USA). A p-value less than 0.05 was considered statistically significant.

Results:

Treatment of Tumors with Nanoparticle-Microbubbles Containing Cabazitaxel

This study was executed as a proof-of-principle, to evaluate whether the increased delivery of NPs to the tumor tissue would be sufficient to improve treatment with encapsulated cytostatic drugs.

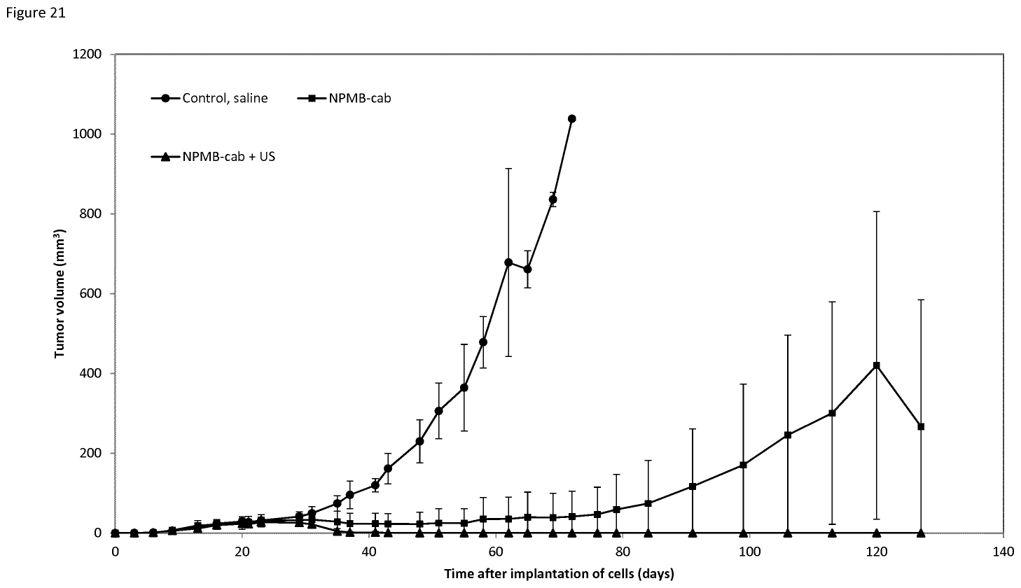

The average tumor growth for the 3 treatment groups is shown in FIG. 21.

Untreated animals (saline) showed a continuous tumor growth and were sacrificed at day 62, 69 and 72 after implantation when the tumors reached 15 mm. The group treated with NPMB encapsulating cabazitaxel showed reduced tumor growth compared to the non-treated animals, and all animals responded to treatment, but with large variations in tumor volume between the animals. The tumors started regrowing approximately 80 days after implantation (50 days after treatment end). One animal was sacrificed at day 120 when the tumor reached 15 mm, and the two other were still alive at the end of the study, with tumors of 13 and 4.5 mm in length. The group treated with FUS in addition to NPMB with cabazitaxel showed a larger reduction in tumor growth, and from day 48, all animals were in complete remission. At the end of the study, approximately 100 days after end of treatment, all animals were still alive and in complete remission (see FIG. 21).

The animals did not lose any weight due to the treatment, neither the control animals nor the animals treated with encapsulated cabazitaxel and FUS.

### Example 8

### Treatment of Orthotopic Breast Cancer (67NR-cells)

A proof of concept experiment was designed to explore differences in effect achieved with the delivery system comprising NP-stabilized MB with ultrasound-mediated delivery of NPs compared with co-injection of NP and SonoVue and ultrasound-mediated delivery. In this experiment, cabazitaxel loaded PEBCA-stabilized MBs, produced as described in Example 6, were used.

Tumor growth as a function of time was compared for mice receiving repeated treatments, and cabazitaxel uptake in tumors is compared for mice receiving only one treatment and sacrificed 6 hours after the treatment.

The composition with NP-stabilized MBs were administrated in concentration of 5,65E+08 (Mean size 2,91).

A total of 30 female balb/c mice were given an injection of 20 ul 67NR tumor cells (500.000 cells) in the mammary fat pad on day 1. Cells were grown and prepared by Shalini Rao and injections were given by Tonje Steigedal. Sixteen (16) of the mice were included in the treatment study, given three injections of NP/NPMB containing cabazitaxel on different days, eight (8) were injected with NP and NPMB only once and sacrificed 6 hours after injection, five (5) were used for testing sonications at different MIs and one (1) had to be sacrificed on day 7 because of poor health condition (stress and low body weight).

Treatment Study

| Groups | Number of animals |
|---|---|
| 1: Control | N = 4 |
| 2: NP + SonoVue + US | N = 6 |
| 3: NPMB + US | N = 6 |

The mice included in the treatment study was given the same treatment on three occations, day 8, day 12 and day 16 after inoculation of tumor cells. On day 7 and 8, all mice were examined and those who had the largest tumors were selected for the treatment study.

Ultrasound

We used the Imasonics 1 MHz transducer in combination with the new E&I 50 dB amplifier and the Agilent signal generator. The mice were placed at a distance of 20 cm from the transducer surface (farfield), and the 3 dB beam width was 9-10 mm. To achieve a mechanical index (MI) of 0.5, we used 270 mVpp as input to the 50 dB amplifier.

MI=0.5

Burst: 10.000 cycles

PRF=0.5 Hz

Duration: 4 minutes

Dosage of Cabazitaxel

The batch BC-1 with nanoparticles with cabazitaxel was used for this experiment. The amount of NP in the NPMB solution corresponded to a concentration of 1 mg cabazitaxel per ml NPMB. This would result in a dose of 0.2 mg in an injection of 200 ul NPMB, hence 10 mg/kg in a mouse of 20 g. Since the bubble concentration of NPMB was very high (similar or higher than SonoVue), we decided to reduce the amount of NPMB to 150 ul, so that the total number of injected bubbles would be the same for group 2 and 3. The total dose of cabazitaxel given in each treatment was hence 0.15 mg corresponding to a dose of 7.5 mg/kg for a 20 g mouse. The BC-1 solution was diluted 1:3, adding lml of saline to a vial containing 0.5 ml BC-1. This resulted in a concentration of 3 mg/ml, hence an injection of 50 ul contained 0.15 mg cabazitaxel.

Treatments

Control: Mice were anestetized by 200 ul of injeciton anastesia (sc) and woken up by 200 ul antidote and put in recovery rack until the next morning. No injections were given.

NP+SonoVue+US: Mice were anestetized by 200 ul of injeciton anastesia (sc). Venflon was placed in the lateral tail vein and the mouse was placed on top of the water tank. 50 ul of NP was injected followed by 150 ul of SonoVue (injected during 5-7 seconds). The ultrasound was turned on just before the SonoVue injection started and the timer started when the injections was finished. The mice were woken up by 200 ul antidote (sc) shortly after the treatment and put in recovery racks until the next morning.

NPMB+UL: Mice were anestetized by 200 ul of injeciton anastesia (sc). Venflon was placed in the lateral tail vein and the mouse was placed on top of the water tank. 150 ul of NPMB was injected during 5-7 seconds. The ultrasound was turned on just before the NPMB injection started and the timer started when the injections was finished.

The mice were woken up by 200 ul antidote (sc) shortly after the treatment and put in recovery racks until the next morning.

Tumor Growth and Weight

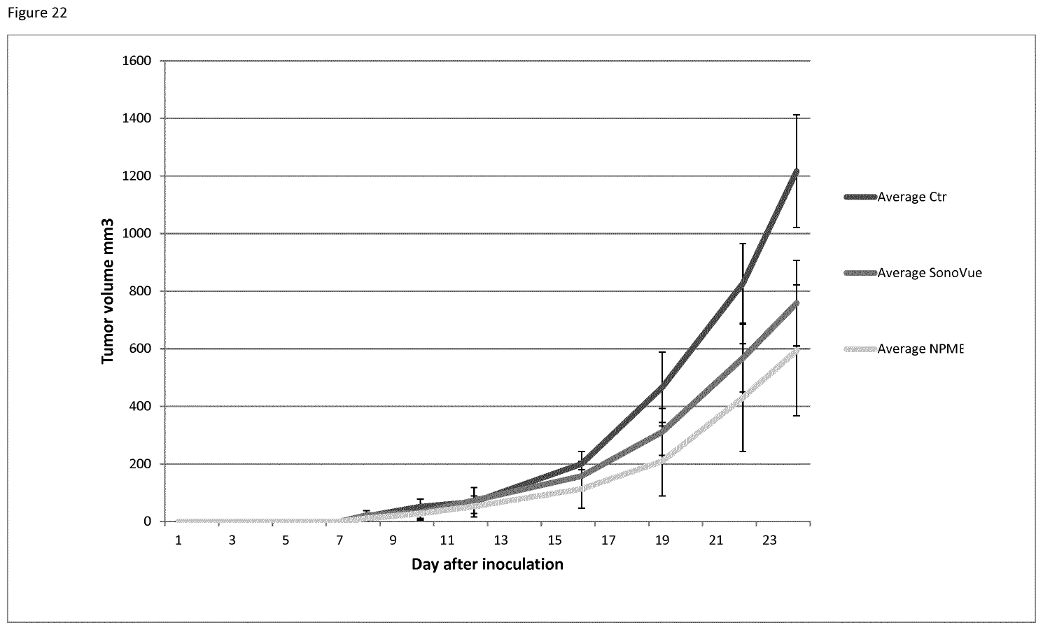

Tumors were measured with caliper on day 8, 10, 12, 16, 19, 22 and 24. Results are shown in FIG. 22.

The four largest tumors are all in the control group, and three smallest are in the NPMB group. The tumors in the NP+SonoVue and in the NPMB groups are similar in size compared to the smallest control tumors.

On day 24 all the mice were sacrificed and the tumors were dissected and weighed. Results showed that the mean of the NPMB group is smaller than the SonoVue-group, however some overlap is seen between the various groups.

### Example 9

### NP-Stabilized MBs for Treatment of Glioma

A glioma cell line was injected intra-cranially in NOD/SCID mice. The glioma was demonstrated to be invasive and the mice had an intact BBB, making it a good model to evaluate the ability of the drug delivery system to cross the BBB and the effect of NPMB and US on tumor growth in the central nervous system.

Tumor growth was monitored weekly with MRI. The tumors were imaged from four weeks post implantation, and treatment was started approximately six weeks post implantation. An MR-FUS system was used to treat the mice 3 times over a period of three weeks. Prior to treatment, the MR-FUS system settings were optimized.

Mice were divided into 4 groups: group 1 was control and did not receive any treatment, group 2 was injected with cabazitaxel alone, group 3 was injected with cabazitaxel together with NPMBs and group 4 was injected with cabazitaxel-loaded NPMBs. Cabazitaxel-loaded PEBCA-stabilized MBs, produced as described in Example 6, were used, To group 3 and 4 US was applied in an area covering the tumor (4 positions 1.2 mm apart moving on a motorized stage). The ultrasound settings used were: 1.2 MHz, 0.38 MPa, 10 ms bursts, 4 minutes, each position was sonicated once every second. The NPMBs were injected in two boluses, the first at treatment start and the second 2 minutes into the treatment. The nanoparticles were fluorescently labelled to be able to track them by fluorescence microscopy.

Four read-outs were used to evaluate the treatment: 1) Tumor growth; 2) quantification of cabazitaxel in tumors (by mass spectrometry), 3) NP uptake in tumors (by confocal laser scanner microscopy of tumor sections); 4) Histology of tumor tissue.

Results:

After the treatment-studies were completed, tumor size in the different groups were observed. The observation revealed a significant decreased tumor growth in the group treated with cabazitaxel-loaded NPMB with US compared with the controls. The results demonstrate the ability of cabazitaxel-loaded NP to penetrate the BBB when used in a delivery system according to the invention, as well as treatment effects of the delivery system on intracranial glioma.

1. A drug delivery method comprising
systemically administering a drug delivery system, and
generating an acoustic field at a release site to mediate the delivery of said drug delivery system to a target site,
wherein the drug delivery system comprises
a gas-filled microbubble,
a plurality of nanoparticles associated with the gas-filled microbubble, free nanoparticles, and
at least one therapeutic agent associated with at least one of the associated nanoparticles and at least one of the free nanoparticles,
wherein the nanoparticles are poly alkyl cyanoacrylate (PACA) nanoparticles.

2. The drug delivery method according to claim 1, wherein the nanoparticles associated with the gas-filled microbubble are surface-associated to the gas-filled microbubble.

3. The drug delivery system according to claim 1, wherein the at least one therapeutic agent is loaded within the nanoparticles.

4. (canceled)

5. (canceled)

6. (canceled)

7. The drug delivery method according to claim 1, wherein the nanoparticles associated with the gas-filled microbubbles stabilizes the microbubbles.

8. The drug delivery method according to claim 1, wherein the nanoparticles further compriseing at least one targeting agent.

9. The drug delivery method according to claim 1, further comprising a pharmaceutically acceptable carrier.

10. The drug delivery method according to claim 1, wherein the nanoparticles further are coated with polyethylene glycol (PEG).

11. The drug delivery method according to claim 1, wherein the mean diameter of the gas-filled microbubbles associated with nanoparticles is in the range of 0.5 to 30 □m.

12. The drug delivery method according to claim 1, wherein the therapeutic agent is a chemotherapeutic agent or a chemopotentiator.

13. The drug delivery method according to claim 1, wherein the gas-filled microbubbles is filled with a gas selected from the group consisting of air, perfluorocarbon, —N₂, O₂, and CO₂.

14. The drug delivery method according to claim 1, wherein the acoustic field is generated by ultrasound.

15. The drug delivery method according to claim 1, wherein the microbubbles are destroyable upon application of focused ultrasound thereto.

16. A method for preparing a drug delivery system for use in therapy according to claim 1, comprising:
a) synthesizing the nanoparticles to be loaded with the therapeutic agent;
b) adding the nanoparticles to a solution comprising a surface-active substance; and
c) mixing the solution of b) with a gas to obtain gas-filled bubbles.

17. The method according to claim 16, wherein the microbubbles are stabilized by self-assembly of nanoparticles in a gas-water interface.

18. (canceled)

19. The method according to claim 16, wherein the solution in c) is mixed from 2 seconds to 60 minutes.

20. The method according to claim 16, wherein the solution in c) is mixed at 500 to 50,000 rpm.

21. The method according to claim 16, wherein the surface-active substance is a serum, a protein, a lipid or a surfactant.

22. A composition comprising
a gas-filled microbubble,
a plurality of nanoparticles associated with the microbubble,
at least one free nanoparticle, and
at least one therapeutic agent associated with at least one of the associated nanoparticles and at least one of the free nanoparticles,
wherein the nanoparticles are poly(alkylcyanoacrylate) (PACA) nanoparticles.

23. The composition according to claim 22, wherein the plurality of nanoparticles associated with the gas-filled microbubble are surface-associated to the gas-filled microbubble.

24. The composition according to claims 22, wherein the one or more therapeutic agents associated with the nanoparticle and microbubble are loaded within the nanoparticles.

25. (canceled)

26. (canceled)

27. The composition according to claim 22, wherein the plurality of nanoparticles associated with the gas-filled microbubbles stabilizes the microbubbles.

28. The composition according to claim 22, wherein the nanoparticles further compriseing at least one targeting agent.

29. The composition according to claim 22, further comprising a pharmaceutically acceptable carrier.

30. The composition according to claim 22, wherein the nanoparticles further are coated with polyethylene glycol (PEG).

31. The composition according to claim 22, wherein the mean diameter of the gas-filled microbubbles associated with a plurality of nanoparticles is in the range of 0.5 to 30 □m.

32. The composition according to claim 32, wherein the one or more therapeutic agents are chemotherapeutic agent or a chemopotentiator.

33. The composition according to claim 22, wherein the gas-filled microbubbles are filled with a gas selected from the group consisting of air, perfluorocarbon, H₂, O₂ and CO₂.

34. A method of treating cancer or diseases in the central nervous system comprising administering a drug delivery system according to claim 1 to a patient in need thereof.
